(19) United States
(12) Patent Application Publication
(10) Pub. No.: US 2025/0263704 A1
(43) Pub. Date: Aug. 21, 2025

(54) NUCLEIC ACID-POLYPEPTIDE NANO-PHARMACEUTICAL COMPOSTION FOR TREATING AND PREVENTING HUMAN PAPILLOMA VIRUS INFECTION

(71) Applicants: SIRNAOMICS, INC., Gaithersburg, MD (US); SIRNAOMICS BIOPHARMACEUTICALS (SUZHOU) CO., LTD., Suzhou (CN); SIRNAOMICS BIOPHARMACEUTICALS (GUANGZHOU) CO., LTD., Guangzhou (CN)

(72) Inventors: Alan Lu, Suzhou (CN); Deling Wang, Suzhou (CN); Shenggao Tang, Guangzhou (CN); Jun Xu, Suzhou (CN); Patrick Y Lu, Suzhou (CN); Xianbin Yang, Suzhou (CN); Weiwei Tian, Suzhou (CN); David Evans, Gaithersburg, MD (US)

(21) Appl. No.: 18/561,596

(22) PCT Filed: May 24, 2022
(86) PCT No.: PCT/CN2022/094631

§ 371 Date: Nov. 16, 2023

(30) Foreign Application Priority Data
May 28, 2021 (CN) 202110592224.6

(51) Int. Cl. C12N 15/113

(52) U.S. Cl. CPC C12N 15/1131; C12N 2310/14; C12N 2310/31; C12N 2310/321; C12N 2310/351

(57) ABSTRACT

Disclosed is a nucleic acid-polypeptide nano-pharmaceutical composition for treating and preventing human papilloma virus infection. A small interfering nucleic acid siRNA molecule used for inhibiting and treating various diseases caused by a HPV infection can block the virus replication life cycle by means of targeted inhibition of the expression of the HP16/18 key gene, reduce a viral infection and finally remove viruses. A pharmaceutical composition based on the siRNA molecule comprises a siRNA molecule and another molecule, including a siRNA molecule for inhibiting PD-1/PD-L1, a small molecule compound against a HPV infection, a therapeutic mRNA/neoantigen vaccine, and the like. The siRNA molecule and other anti-HPV drugs are coupled by means of a specific chemical bond to form a new coupled molecule, and the composition further includes a pharmaceutically acceptable polypeptide polymer nano-introduction carrier. In some embodiments, the carrier is a histidine-lysine polypeptide polymer nanocarrier.

### CROSS-REFERENCE TO RELATED APPLICATIONS

This application is the U.S. National Stage of PCT/CN2022/094631 filed on May 24, 2022, which claims priority to Chinese Patent Application 202110592224.6 filed on May 28, 2021, the entire content of both are incorporated herein by reference in their entirety.

### SEQUENCE LISTING

The contents of the electronic sequence listing (entitled SRNI0001PASEQ_ST25.txt, created 2024 Apr. 19 and having a file size of 61523 bytes) is herein incorporated by reference in its entirety.

### TECHNICAL FIELD

The present invention belongs to the technical field of new drugs, and involves a nucleic acid polypeptide nano-pharmaceutical composition for treating and preventing human papillomavirus infection.

### BACKGROUND

### HPV and Cervical Cancer

Human papillomavirus (HPV) is a group of enveloped DNA viruses, which now has over 100 different species, and is the most common sexually transmitted (ST) infection in adults worldwide. In 1976 Harald zur Hausen from Germany published the hypothesis that human papillomavirus plays an important role in the cause of cervical cancer tissue^{[1]}. In 1983 and 1984, zur Hausen and his collaborators identified HPV 16 and HPV 18 in cervical cancer^{[2-4]}. Zur Hausen was awarded the Nobel Prize for Physiology and Medicine in 2008, because of his contribution. It is estimated that over 80% of US women by age 50 will be infected with at least one HPV strain^{[5]}. In addition, there are 490,000 new cases of cervical cancer every year worldwide, resulting in 270,000 deaths. In the United States, there are 250,000 to 1 million women who develop cervical dysplasia each year, which may lead to 11,000 women further developing cervical cancer and to 4,000 deaths^{[6]}. Among the 19 “high-risk” HPVs which may lead to cervical cancer, HPV 16 and 18 count for about 70% of the cases^{[7]}.

HPVs have a circular genome of about 8 kb, with three major regions in the genome, namely early genes (E6, E7, E1, E2, E4 and ES), the late genes (L1 and L2) and the longer control region (LCR) between L1 and L6. FIG. 1 shows the characteristic HPV genome organization, using the medically important HPV-16 as the model. The early transcripts ending at 4215 encode the 6 early genes, while the late transcripts ending at 7221 encode the two late genes. E6 and E7 are early transcribed cancer transforming proteins because they can inactivate tumor suppressor proteins p53 (inactivated by E6) and pRb (inactivated by E7)^{[8]}.

Although the US FDA has approved two HPV vaccines (described in detail late), there is still a high demand for HPV therapeutics. However, there is no effective treatment on the market yet^{[9]}. The present invention describes HPV therapeutics by siRNAs complexed with histidine-lysine polymers.

### HPV Vaccine

In 2006, the US FDA approved GARDASIL®, an HPV vaccine produced by Merck, which is composed of a hollow virus-like particle (VLP) assembled from recombinant HPV coat proteins and which targets HPV 16, 18, 6 and 11. The vaccine is intended for use in women and girls. Later, according to extended studies, it was reported that GARDASIL® is also effective in preventing genital warts in males. The GARDASIL® for use in men and boys was approved by the FDA on Oct. 16, 2009. In October 2009, the FDA also approved a second HPV vaccine, CERVARIX®, targeting HPV 16 and HPV 18, produced by GlaxoSmithKline^{[10]}. In June 2015, Merck & Co received great news in European supervision. Its super human papillomavirus (HPV) vaccine, GARDASIL®9 (9-valent HPV vaccine), was approved by the European Commission. The vaccine is the successor of GARDASIL®4 (4-valent HPV vaccine), covering 9 genotypes of HPV, and has the potential to prevent about 90% of cervical cancer, vulvar cancer, vaginal cancer, and anal cancer. Previously, the FDA has approved GARDASIL®9 in December 2014. The industry predicts that GARDASIL®9 will replace GARDASIL®4 as the world's best-selling HPV vaccine, with sales peaking at $1.9 billion.

Public health officials in developed countries and regions (such as Australia, Canada, Europe, and the United States) recommend vaccination of young women against HPV to prevent cervical cancer and genital warts, and to reduce the painful and costly treatments for cervical dysplasia caused by HPV infections. It is recommended that all women and girls who are not exposed to HPVs between the ages of 9 to 25 should get an HPV vaccination^{[11]}. However, many women and girls are not vaccinated against HPV for various reasons. In the US, only about one-quarter of girls got HPV vaccination because most families worried about either the effectiveness or the side effects of the vaccine^{[12]}. In addition, HPV vaccines are not very easy to get access to in third world countries. In Kenya, as an example, the cost of vaccination is over the average annual income of a family^{[13]}. In addition, many women have been exposed to HPV^{[14]}, and the treatment of HPV is required.

Development of siRNA and Novel Therapeutics Targeting HPV 16 and HPV 18

RNA interference was originally discovered in plants but quickly proved to be a universal process covering both low and high biological species. This is an efficient process in which double-stranded RNA duplexes were generated and lead to the recognition, binding and degradation of specific target messenger RNAs^{[15]}. In recent years, RNAi has been used in various biological studies, and applied in therapeutic development as well^{[16]}. So far, at least 15 RNAi therapies have been developed, and these therapies are in different stages of clinical trials or have completed clinical trials^{[17]}, and four new RNAi drugs have been approved to enter the market in the United States and Europe.

After in silico screening by computer software, some candidate siRNA sequences targeting HPV 16 E7 and HPV 18 E7 were obtained. After these sequences were chemically synthesized, the biological functions of candidate small interfering nucleic acid sequences were further verified and screened using in vitro cell systems and in vivo HPV animal models. Through in vitro cytology experiments, it was confirmed that the simultaneous introduction of the corresponding HPV 16 E7 siRNA and HPV 18 E7 siRNA can significantly inhibit the mRNA expression level of the target gene, and a good therapeutic effect has been obtained in the corresponding cottontail rabbit animal model. Therefore, the use of chemically synthesized HPV 16 E7 and HPV 18 E7 modified siRNA-small molecule conjugates to treat HPV and HIV and/or HSV infection etc. opens up a new therapeutic approach. The drug with a clear mechanism of action, a clear target, and a unique and effective delivery system is a novel drug different from traditional small molecule or monoclonal antibody drugs.

Histidine-Lysine Co-Polymer (HKP) for siRNA Delivery In Vivo

Although RNAi has offered a very attractive technology to be developed into innovative therapeutics, many of the projects haven't succeeded. The failure of most projects is attributed to the stability issue of siRNA^{[16]}. Naked siRNAs have to be modified to protect them from degradation, or to be packed with other molecules either to facilitate cell entry or to be functional to decrease target gene expression^{[18]}. Therefore, the development of delivery methods has been one of the most important areas in the research and development of siRNA therapeutics^{[19]}.

A histidine-lysine co-polymer (HKP) is a positively charged branched polymer (FIG. 2) which has been successfully used for in vivo delivery of plasmid DNA and siRNA. We have used HKP for in vivo introduction of nucleic acids in various tissue types, including skin scars, livers, lungs, tumors, eyes, and brains.

### SUMMARY

The technical problem to be solved by the present invention is to provide a pharmaceutical composition that can be used for preparing targeted drugs for treating HPV infections.

In order to solve the above technical problems, the present disclosure takes the following technical protocols:

A nucleic acid polypeptide nano-pharmaceutical composition, it includes siRNAs of HPV 16-E7 and HPV 18-E7, conjugates of these siRNAs and small molecule drugs, and a pharmaceutically acceptable carrier suitable for delivering drugs in vivo, as well as a nano-drug consisting of the carrier and nucleic acids.

It includes siRNAs targeting HPV 16 E7 and HPV 18 E7, and a carrier suitable for in vivo introduction, and the carrier is the histidine-lysine co-polymer (HKP) or modification thereof. Specifically, the HPV 16 E7 siRNA includes HPV 16 E7 siRNA-45#, and the sequence of the HPV 16 E7 siRNA-45# is 5′-GCACCCUGGGCAUCCUGUGCCCCAU-3′ (SEQ ID NO: 26).

The HPV 16 E7-45#siRNA is double-stranded, easy to degrade, needs to be dissolved in RNase-free treating water, and can be packed by adding a positively charged carrier to improve stability.

The composition includes the pharmaceutically acceptable carrier which can be selected from carriers including, but are not limited to: physiological saline, sugar solutions, polypeptides, polymers, lipids, cream gels, micellar materials, metal nanoparticles, dendrimers, and HK polymers.

The positively charged carrier is the histidine-lysine co-polymer (HKP).

Such co-polymer is described in several patents of U.S. Pat. Nos. 7,070,807 B2, 7,163,695 B2, and 7,772,201 B2, and the entire content is incorporated herein by reference. Preferably, the HKP carrier is H3K4b, H3K(+H)4b, H2K4b or H3K(+N)4b. These HKPs have a lysine backbone, and four branches of the lysine backbone contain multiple repeated histidine, lysine or asparagine.

The HPV 16 E7 siRNA includes HPV 16-CRPV E7 siRNA-43#, and the sequence of the HPV 16-CRPVE7 siRNA-43# is 5′-GGAAGACCUGCUGAUGGGCACCCU-3′ (SEQ ID NO: 24).

The HPV 16-CRPVE7 siRNA-43# is a siRNA sequence designed according to the mRNA homologous sequence of CRPV E7 (namely cotton rabbit papillomavirus) and HPV 16 E7.

The HPV 18 E7 siRNA includes HPV 18 E7 siRNA-44#, and the sequence of it is 5′-GCUCAGCAGACGACCUUCGAGCAUU-3′ (SEQ ID NO: 7).

The HPV 18 E7 siRNA includes HPV 18 E7 siRNA-44#, and the sequence of it is 5′-GCUGUUUCUGAACACCCUGUCCUUU-3′ (SEQ ID NO: 8).

The siRNA molecules include HPV 16-CRPVE7 siRNA-43# and HPV 18 E7 siRNA-44#. They are mixed into double-target siRNA cocktail which can be used for enhancing effects of the siRNA molecule resisting HPV, HIV and/or HSV infection.

The siRNA molecules include HPV 16-CRPVE7 siRNA-43# and HPV 18 E7 siRNA-46#. They are mixed into double-target siRNA cocktail which can be used for enhancing effects of the siRNA molecule resisting HPV, HIV and/or HSV infection.

The siRNA molecules include HPV 16 E7 siRNA-45# and HPV 18 E7 siRNA-44#. They are mixed into double-target siRNA cocktail which can be used for enhancing effects of the siRNA molecule resisting HPV, HIV and/or HSV infection.

The siRNA molecules include HPV 16 E7 siRNA-45# and HPV 18 E7 siRNA-46#. They are mixed into double-target siRNA cocktail which can be used for enhancing effects of the siRNA molecule resisting HPV, HIV and/or HSV infection.

The siRNA molecules include HPV 16-CRPVE7 siRNA-43# and HPV 18 E7 siRNA-44#, as well as HPV 18 E7 siRNA-46#. They are mixed into three-target siRNA cocktail which can be used for enhancing effects of the siRNA molecule resisting HPV, HIV and/or HSV infection.

The siRNA molecules include HPV 16 E7 siRNA-45# and HPV 18 E7 siRNA-44#, as well as HPV 18 E7 siRNA-46#. They are mixed into three-target siRNA cocktail which can be used for enhancing effects of the siRNA molecule resisting HPV, HIV and/or HSV infection.

The siRNA molecules include HPV 18 E7 siRNA-44# and HPV 16-CRPVE7 siRNA-43#, as well as HPV 16 E7 siRNA-45#. They are mixed into three-target siRNA cocktail which can be used for enhancing effects of the siRNA molecule resisting HPV, HIV and/or HSV infection.

The siRNA molecules include HPV 18 E7 siRNA-46# and HPV 16-CRPVE7 siRNA-43#, as well as HPV 16 E7 siRNA-45#. They are mixed into three-target siRNA cocktail which can be used for enhancing effects of the siRNA molecule resisting HPV, HIV and/or HSV infection.

A pharmaceutical composition for preventing or treating HPV infection, and the active ingredients of the pharmaceutical composition may include siRNA molecules for inhibiting HPV replication and the other molecule(s). The another molecule(s) may include one or more of siRNA molecule(s) for inhibiting human immune regulation related genes, an anti-HPV small molecule compound, a cervical cancer mRNA vaccine, or an anti-HPV monoclonal antibody.

The siRNA molecules for inhibiting human immune regulation related genes are siRNA molecules for inhibiting immune checkpoints, including but are not limited to: siRNA molecules for inhibiting PD-1, siRNA molecules for inhibiting PD-L1, siRNA molecules for inhibiting LAG-3, siRNA molecules for inhibiting TIM-3, siRNA molecules for inhibiting VISTA, siRNA molecules for inhibiting TIGIT, and siRNA molecules for inhibiting CTLA-4/B7.

The anti-HPV small molecule compound(s) are selected from one or more of Cidofovir and Brincidofovir, or one or more of artesunate and dihydroartemisinin.

The cervical cancer mRNA vaccine is a messenger RNA vaccine that uses HPV gene fragments to encode specific proteins so as to induce a human body to form a protection effect against HPV infection.

The anti-HPV monoclonal antibody is a therapeutic antibody drug for treating various diseases caused by HPV infection.

In the composition of the present invention, the histidine-lysine co-polymer (HKP) is a positively charged branched histidine-lysine polymer, and is used for nucleic acid delivery in various tissue types.

The modifier of the histidine-lysine co-polymer is a branched histidine-lysine polymer (HKP+H) with a histidine added, which is used for nucleic acid delivery in various tissue types and inducing extremely low immune and inflammatory responses.

The histidine-lysine co-polymer adopts H3K4b, which consists of three lysine cores and four branches. Each of the four branches includes a large number of repeated histidines and lysines, and the specific structure is shown in FIG. 2.

The modifier of the histidine-lysine co-polymer adopts H3K(+H)4b, and its specific structure is to add a histidine on the branch of H3K4b, that is, the structure of the H3K(+H)4b is to replace the side strand R in FIG. 2 with R=KHHHKHHHKHHHHKHHHK (SEQ ID NO: 303).

Provided is a nucleic acid polypeptide nano-pharmaceutical composition. The nano-drug comprises a pharmaceutically acceptable carrier, and the carrier mixes siRNA molecules at a specific nitrogen-to-phosphorus (N:P) ratio to form a nano-drug with a specific size.

Provided is the nano-pharmaceutical composition prepared from HKP and a siRNA drug or the composition based on the siRNA drug, where the HKP carries a positive charge, while siRNA, a composition of siRNA and siRNA, a composition of siRNA and mRNA vaccines, etc. carry a negative charge. When an HKP aqueous solution is mixed with the siRNA or the composition based on the siRNA drug at a specific mass ratio (such as 4:1), the nanoparticles will be formed through self-assembling. The average diameter of the nanoparticles is in the range of 50-300 nm, and further preferably, the size of the nanoparticles is 80-150 nm.

The N:P mass ratio of the carrier to the small nucleic acid molecule siRNA is 16:1-1:8.

The N:P mass ratio of the carrier to the small nucleic acid molecule siRNA is greater than or equal to 4:1.

A single siRNA molecule binds the mRNA encoded by one HPV gene. The 20-40 nucleotide pairs of HPV 16 or HPV 18 are inserted into the end of the E7 gene of cotton tail rabbit papillomavirus in the same “reading frame” to form a fusion protein, and the 20-40 nucleotide pairs can be used as an attack sequence site of siRNA.

The fusion virus formed by such fusion protein can infect the skin of cotton tail rabbits and form normal infection spots. Changes in the infection spots will be indicative of the efficacy of small interfering nucleic acid therapy.

The HKP of the present invention is entrusted to an outsourcing company and synthesized according to the patented technology owned by the inventor. FIG. 3 explains the specific steps of HKP synthesis.

Provided is an application of the small interfering nucleic acid pharmaceutical composition to preparation of targeted drugs for treating HPV infection.

The second aspect of the present invention is to provide a pharmaceutical composition for preventing or treating HPV virus infection. The active ingredients of the pharmaceutical composition include siRNA molecules targeting HPV viruses and small molecule compounds resisting the HPV viruses.

The nucleotide analogue(s) for inhibiting the HPV viruses are selected from one or more of Cidofovir and Brincidofovir.

The artemisinin derivative(s) are selected from one or more of artesunate and dihydroartemisinin.

Small nucleic acid siRNAs comprise special 2′-OMe, 2′-F, 2′-MOE, sulfur-modified phosphate backbones, base modifications, antisense and sense 5′ end modifications, and other chemically modified small nucleic acids to improve the stability of the small nucleic acid siRNAs and reduce the off-target effect and immune response of the small nucleic acid siRNAs.

The modified small nucleic acid siRNAs include 19+2 double strands, 21+23 double strands, etc. with a special asymmetric structure.

### DESCRIPTION OF DRAWINGS

FIG. 1A is an HPV genome. The amplification of the E7 gene is shown at the bottom, and three sequences to be inserted into the cotton tail rabbit genome are marked in black.

FIG. 1B shows a wild-type E7 gene targeting HPV 16 and the siRNA targeting heterozygous E7 genes from HPV 16 and CRPV. The red represents the CRPV sequences for replacing the corresponding HPV 16 fragments. The yellow area in CRPV E7 siRNAs reflects the codon optimization results.

FIG. 1C is the gene construction of a chimeric human rabbit papillomavirus (cH-RPV). Three epitope sequences A, B and C from the HPV 16 E7 gene were inserted in the same reading frame at the end of the CRPV E7 gene.

FIG. 2 is a schematic diagram of the structure of a histidine-lysine co-polymer and a histidine modification added to the side strand, where R represents the amino acid sequence of the four branched side strand.

FIG. 3 shows the synthesis steps of HKP.

FIG. 4 shows the process of forming a complex from siRNA and HKP (left Fig.) and a cotton tail rabbit skin infection papillomavirus model (SIRAM) (right Fig.).

FIG. 5 is the screening result of HPV 16 E7 siRNA in SiHa cells (the mRNA expression of a target gene detected by real-time fluorescence quantitative method).

FIG. 6 is the screening result of HPV-CRPV16 E7 siRNA in SiHa cells (the mRNA expression of a target gene detected by real-time fluorescence quantitative method).

FIG. 7 is the screening result of HPV 18 E7 siRNA in Hela cells (the mRNA expression of a target gene detected by real-time fluorescence quantitative method).

FIG. 8 shows the inhibitory effect of siRNA at the protein expression level of an HPV 16 E7 gene in the SiHa cells detected by a Western method. Western blot (left) and quantitative data (right) show that siRNA can effectively reduce the expression of E7 proteins, and the order of knockdown effects is −45>−43>−44>−37, which is consistent with the result of real-time fluorescence quantitative trials.

FIG. 9 shows the therapeutic effect of siRNA (CRPV-43) in the cH-RPV cotton tail rabbit model, and the results show that the siRNA has a good inhibitory effect on the growth of rabbit skin warts (L). The data is shown on the right (R).

FIG. 10 shows a summary of data on different siRNA treatments for cH-RPV, with effective siRNAs highlighted.

FIG. 11 shows the effect 1 of HPV 16 and HPV 18 siRNA combination therapy detected by the in vitro real-time fluorescence quantitative method. That is to say, the HPV 16-CRPV-43#siRNA and the HPV 18-44#siRNA or the HPV 18-46#siRNA were transfected into Siha cells with the ratio of 1:2 and 1:1 or 2:1; and then the real-time quantitative PCR method was used for detecting the expression of the corresponding target gene (HPV 16 E7) mRNA, so as to determine the combined effects of the two siRNAs.

FIG. 12 shows the effect 2 of HPV 16 and HPV 18 siRNA combination therapy detected by the in vitro real-time fluorescence quantitative method. That is to say, the HPV 16-45#siRNA and the HPV 18-44#siRNA or the HPV 18-46#siRNA were transfected into Siha cells with the ratio of 1:2 and 1:1 or 2:1; and then the real-time quantitative PCR method was used for detecting the expression of the corresponding target gene (HPV 16 E7) mRNA, so as to determine the combined effects of the two siRNAs.

FIG. 13 shows the effect 3 of HPV 16 and HPV 18 siRNA combination therapy detected by the in vitro real-time fluorescence quantitative method. That is to say, the HPV 18-44#siRNA and the HPV 16-CRPV-43#siRNA or the HPV 16-45#siRNA were transfected into Hela cells with the ratio of 1:2 and 1:1 or 2:1; and then the real-time quantitative PCR method was used for detecting the expression of the corresponding target gene (HPV 18 E7) mRNA, so as to determine the combined effects of the two siRNAs.

FIG. 14 shows the effect 4 of HPV 16 and HPV 18 siRNA combination therapy detected by the in vitro real-time fluorescence quantitative method. That is to say, the HPV 18-46#siRNA and the HPV 16-CRPV-43#siRNA or the HPV 16-45#siRNA were transfected into Hela cells with the ratio of 1:2 and 1:1 or 2:1; and then the real-time quantitative PCR method was used for detecting the expression of the corresponding target gene (HPV 18 E7) mRNA, so as to determine the combined effects of the two siRNAs.

FIG. 15 shows the coupling method of siRNA molecules and nucleotide analogues. As the molecules all contain amino groups, hydroxyl groups and phosphoric acid active groups, phosphoramidite monomers suitable for solid-phase synthesis resulted from molecular modification of the molecules can be directly used for siRNA ligation.

FIG. 16 shows the coupling mode of siRNA molecules and artemisinin derivatives. As the molecules contain carboxylic acid or hydroxyl active groups, siRNA can be linked by an addition reaction.

FIG. 17 shows a general way to couple other drug molecules to one end of siRNA. Through the phosphate groups, specific small molecules for treating HPV infections can be linked to siRNA molecules for inhibiting HPV replication.

FIG. 18 shows a modification mode of siRNA. A shows a schematic diagram of the modification mode of the phosphate backbone or base, and B shows the modification at different positions of siRNA to form 19+2 double strands and 21+23 double strands with special asymmetric structures.

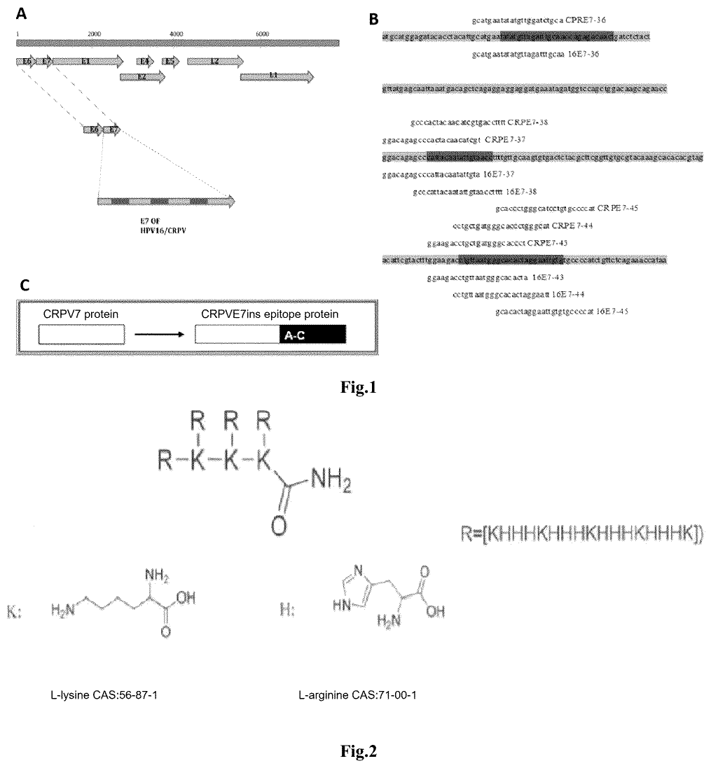

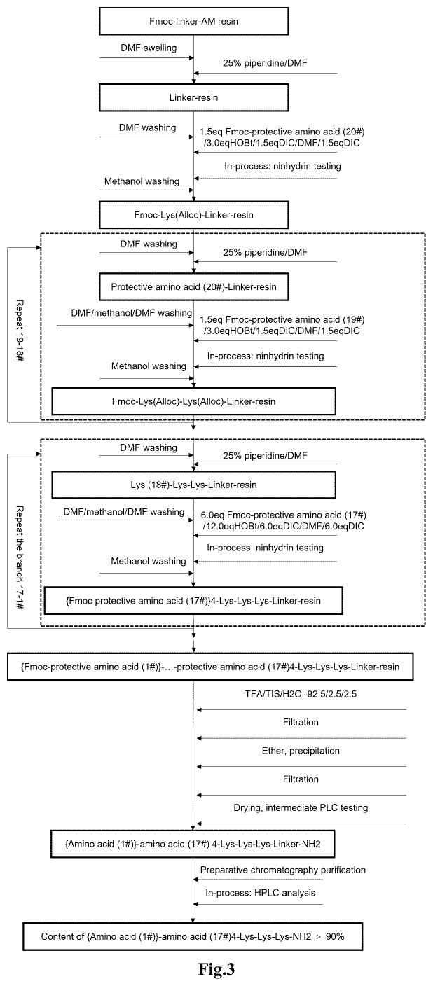

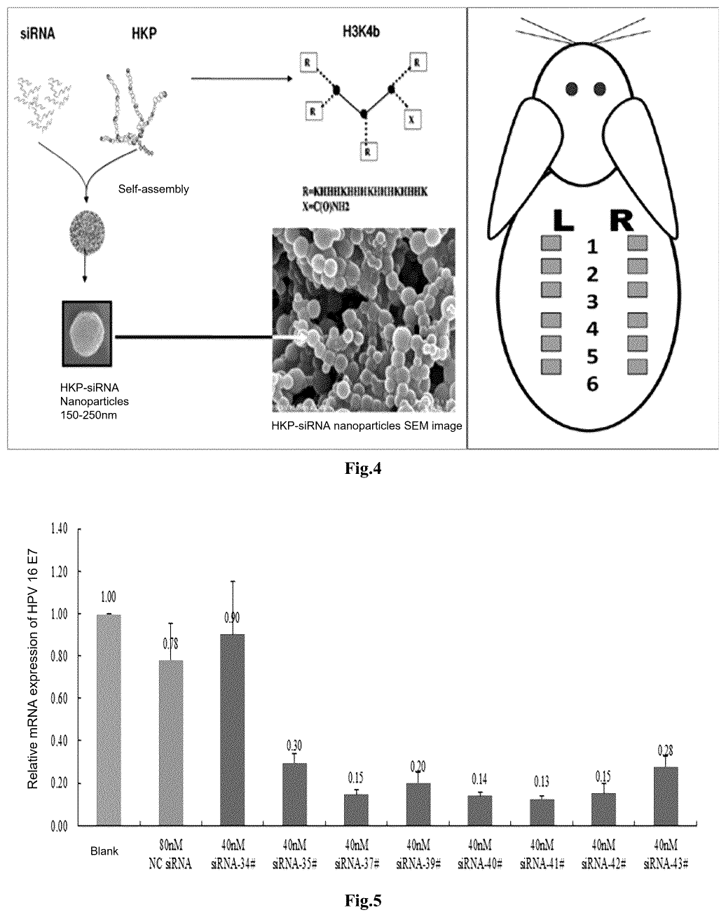

### DETAILED DESCRIPTION OF EMBODIMENTS

The present invention will be further described in detail in combination with specific embodiments, but the present invention is not limited to the following embodiments.

### Embodiment 1. Preparation of Effective siRNA Double Strands Targeting HPV 16-E7, HPV 18-E7, and cH-RPV-E7

In preliminary studies, we have demonstrated that 25 mer siRNAs are the most effective in inhibiting the expression of specific genes. To ensure the efficacy of each siRNA in knocking down target genes, several key features of the siRNA should be considered during in silico design and subsequent in vitro and in vivo trials:
(1) the siRNAs have the optimum thermodynamics for target sequence binding;
(2) the siRNAs have sufficient length for RISC binding;
(3) the siRNAs have eliminated (or added) immune stimulating motifs;
(4) the siRNAs have minimized “Off-Target” potential;
(5) the siRNAs pass through patent search, with no conflict with the current patent; and
(6) the siRNAs have no interaction when multiple sequences are mixed in a cocktail.

In the present disclosure, we designed siRNAs targeting conserved gene sequences, which are shared by as many HPV species as possible, so as to increase the wide applicability of the siRNAs. Furthermore, our preliminary results have demonstrated that 25 mer siRNA is more effective than 21 mer siRNA. We used 25 mer siRNA to design siRNAs targeting an early gene E7. The specific siRNA sequences are as follows:

Design Sequence of HPV 18 E7 siRNA:

HPV18E7-31:
(SEQ ID NO: 1)
GCAUGGACCUAAGGCAACAUUGCAA
HPV18E7-34:
(SEQ ID NO: 2)
GGUUGACCUUCUAUGUCACGAGCAA
HPV18E7-36:
(SEQ ID NO: 3)
GCAAUUAAGCGACUCAGAGGAAGAA
HPV18E7-38:
(SEQ ID NO: 4)
CGAUGAAAUAGAUGGAGUUAAUCAU
HPV18E7-39:
(SEQ ID NO: 5)
CGAGCCGAACCACAACGUCACACAA
HPV18E7-43:
(SEQ ID NO: 6)
GCCAGAAUUGAGCUAGUAGUAGAAA
HPV18E7-44:
(SEQ ID NO: 7)
GCUCAGCAGACGACCUUCGAGCAUU
HPV18E7-46:
(SEQ ID NO: 8)
GCUGUUUCUGAACACCCUGUCCUUU

Design Sequence of HPV 16 E7 siRNA:

HPV16E7-34:
(SEQ ID NO: 9)
GCAUGGAGAUACACCUACAUUGCAU
HPV16E7-35:
(SEQ ID NO: 10)
GGAGAUACACCUACAUUGCAUGAAU
HPV16E7-36:
(SEQ ID NO: 11)
GCAUGAAUAUAUGUUAGAUUUGCAA
HPV16E7-37:
(SEQ ID NO: 12)
GGACAGAGCCCAUUACAAUAUUGUA
HPV16E7-38:
(SEQ ID NO: 13)
GCCCAUUACAAUAUUGUAACCUUUU
HPV16E7-39:
(SEQ ID NO: 14)
GCAAGUGUGACUCUACGCUUCGGUU
HPV16E7-40:
(SEQ ID NO: 15)
GCGUACAAAGCACACACGUAGACAU
HPV16E7-41:
(SEQ ID NO: 16)
CGUACAAAGCACACACGUAGACAUU
HPV16E7-42:
(SEQ ID NO: 17)
GCACACACGUAGACAUUCGUACUUU
HPV16E7-43:
(SEQ ID NO: 18)
GGAAGACCUGUUAAUGGGCACACUA
HPV16E7-44:
(SEQ ID NO: 19)
CCUGUUAAUGGGCACACUAGGAAUU
HPV16E7-45:
(SEQ ID NO: 20)
GCACACUAGGAAUUGUGUGCCCCAU

siRNA Design Sequence for the E7 Genes in cH-RPV (Chimeric Human Rabbit Papillomaviruses):

CRPE7-36:
(SEQ ID NO: 21)
5′-GCAUGAAUAUAUGUUGGAUCUGCA-3′
CRPE7-37:
(SEQ ID NO: 22)
5′-GGACAGAGCCCACUACAACAUCGU-3′
CRPE7-38:
(SEQ ID NO: 23)
5′-GCCCACUACAACAUCGUGACCUUUU-3′
CRPE7-43:
(SEQ ID NO: 24)
5′-GGAAGACCUGCUGAUGGGCACCCU-3′
CRPE7-44:
(SEQ ID NO: 25)
5′-CCUGCUGAUGGGCACCCUGGGCAU-3′
CRPE7-45:
(SEQ ID NO: 26)
5′-GCACCCUGGGCAUCCUGUGCCCCAU-3′

### Embodiment 2. Screening siRNAs in Cell Lines Carrying HPV Genes (FIGS. 5, 6, 7)

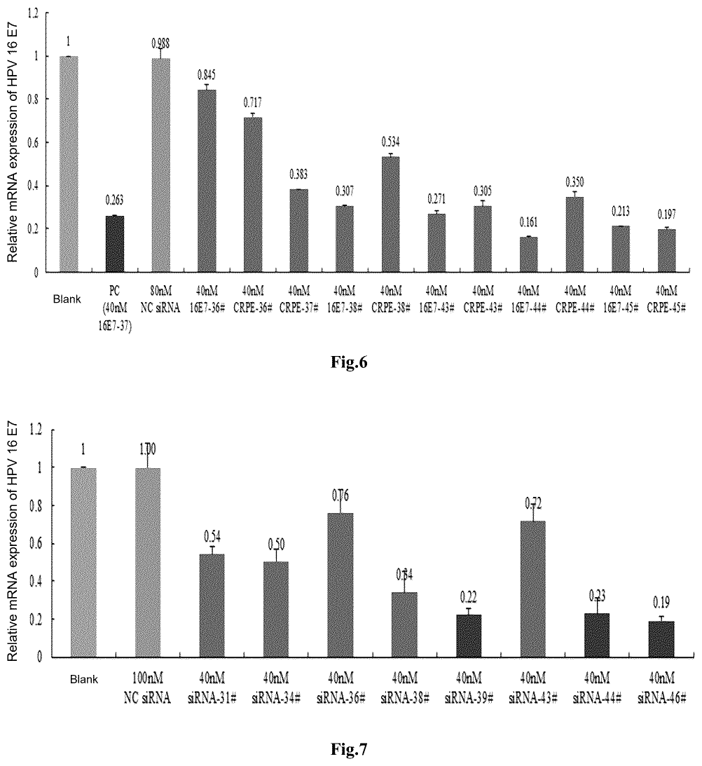

SiHa is a cervical cancer cell line that contains the HPV 16 genome and expresses the oncogene protein E7. The SiHa cell line was used for screening the function of siRNAs targeting the E7 genes in HPV 16 and cH-RPV virus strains. The SiHa cells were cultured with an RPMI 1640 medium containing 10% FBS, 100 U/mL penicillin, and 100 μg/mL streptomycin, and cultured at 37° C. in an incubator containing 10% CO₂. siRNAs were transfected into cells using LipofectAmine 2000 following the manufacturer's instructions. The cells were harvested, and E7 gene expression levels were assessed by qRT-PCR. In addition, the same cell samples were also applied for ELISA and Western analysis. The results in FIGS. 5 and 6 show that the effects of HPV 16 E7 siRNA-37#-40#-41#-42#-44# and cH-RPVE7 siRNA-37#-43#-45# are better.

Similarly, an HPV 18 genome was fused to a cellular genome of HeLa cervical cancer cells to screen siRNAs targeting HPV 18 gene expression. Cells were cultured in the medium similarly described above. siRNA transfection, qRT-PCR, ELISA, and Western followed the same procedure. The results in FIG. 7 show that the effect of HPV 18 E7 siRNA-39#-44#-46# is better.

### Embodiment 3. Western Analysis to Confirm the Knocking Down of E7 Protein Expression by siRNA Sequences

By the Western blot method, we further studied the effect of cH-RPVE7 siRNAs on inhibiting the expression level of E7 proteins. In FIG. 8, the Western blot (left) and quantitative data (right) showed that the potency of the siRNAs reducing E7 protein expression is listed as follows: −45>−43>−44>−37, which fit the results in the qRT-PCR.

### Embodiment 4. The Efficacy of the siRNAs in Skin Infection Rabbit Animal Model (SIRAM)

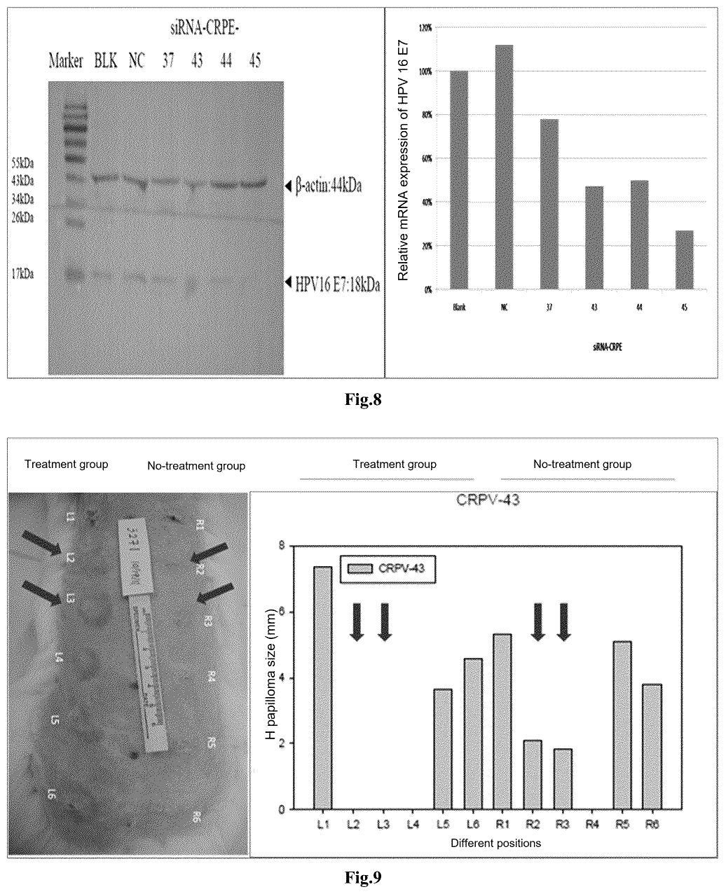

The cotton tail rabbit used in the experiment was CRPV/NZW. In order to test the therapeutic efficacy of the siRNA, we have validated in the in vitro cell screening system. As illustrated in FIG. 9, 6 different wild type and hybrid viruses were inoculated in the NZW rabbit skin. Each animal wore an Elizabeth collar to avoid treatment sites from being disturbed by other animals.

In the preliminary studies, 6 animals were used in the experiments. In each animal, L1-R1, L2-R2, L3-R3, L4-R4, L5-R5, and L6-R6 were challenged with 6 different viruses respectively as illustrated in the FIG. 9. Two weeks after infection, the left sites of the papillomas were treated with corresponding siRNAs, N.C.siRNA and Cidovofir (viral infection small molecule inhibitors) locally for 5 consecutive days. Papilloma outgrowth began to be monitored at week 3 and ended at the termination of the experiment at the end of week 5. Pictures were also taken for record. Right sites are untreated control for the left treated sites. If the siRNA is effective, we should see smaller or no papillomas on the left sites. The viruses that infected L5-R5 sites are more vigorous than those infected sites L2-R2, L3-R3 and L4-R4. L1-R1 infected with wild-type CRPV is used as a specificity control for the siRNAs. Therefore, if an epitope specific siRNA is effective, it should not influence the L1-R1 sites, but the sites that challenged with the viruses containing this epitope, such as L5-R5.

The viruses are described in the following:
L1-R1, wt CRPV DNA 5 μg/site;
L2-R2, CRPV, with HPV 16 E7/A 82-90 fusion viruses;
L3-R3, CRPV, with HPV 16 E7/B 45-57 fusion viruses;
L4-R4, CRPV, with HPV 16 E7/C 11-20 fusion viruses;
L5-R5, CRPV, with HPV 16 E7/82-90 fusion viruses of L2;
L6-R6, CRPV, tandem repeat with HPV 16 E7.

Two weeks after papillomas appeared on the skin or the viruses were infected, we applied different siRNAs to treat papillomas to evaluate the efficacy of the siRNAs. The following are the siRNAs applied on the animals:
Rabbit #3270, siRNA-CRPC-37
Rabbit #3271, siRNA-CRPC-43
Rabbit #3272, siRNA-CRPC-44
Rabbit #3273, siRNA-CRPC-45
Rabbit #3274, siRNA-NC;
Rabbit #3275, Cidofovir, positive control

In the experiment, CRPV-43 treatment inhibited the papilloma growth (FIG. 9).

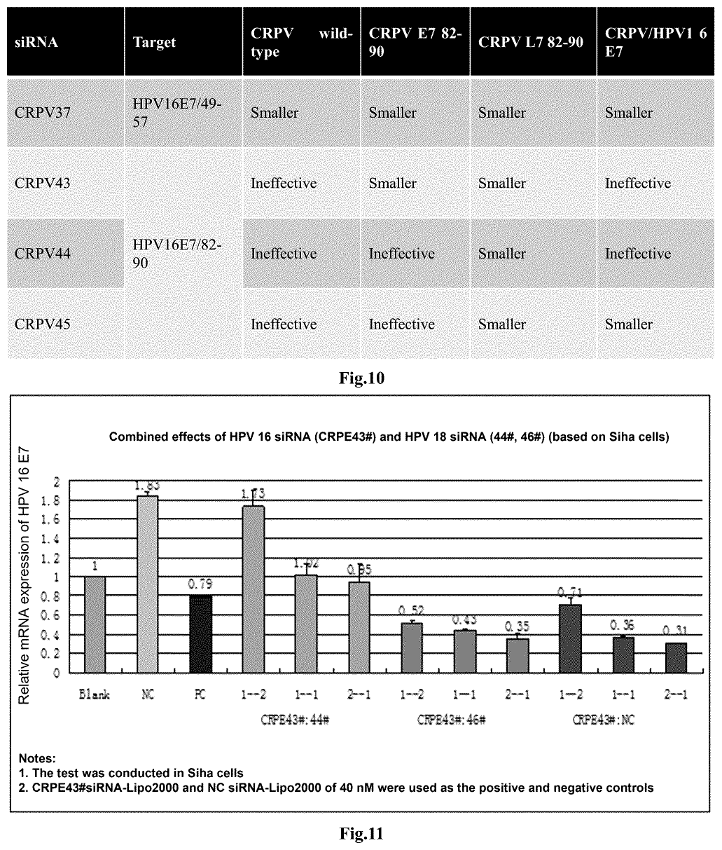

The ability of the siRNAs to against the growth of heterozygous human rabbit papillomavirus (cH-RPV) was summarized in FIG. 10.

### Embodiment 5. Related Experiments of Combined Effects of HPV 16-18 siRNA on Cells In Vitro

Two siRNAs with relatively good transfection effects were selected from HPV 16 and HPV 18 siRNAs respectively, and then HPV 16-18 siRNAs were mixed in different ratios, and simultaneously transfected into Siha cells (specifically expressing HPV 16) and Hela cells (specifically expressing HPV 18), and then the mRNA expression of the corresponding target genes (HPV 16 E7 and HPV 18 E7) was detected by the real-time quantitative PCR method, so as to determine the combined effects of the two siRNAs.

Cell preparation: Hela cells and siha cells and a 12-well cell culture plate with 2× 10⁵ cells/well were prepared the day before.

### Sample Grouping:

| Lipfectamine 2000 was | | | | | | | |
|---|---|---|---|---|---|---|---|
| transfected into the cells in | | | | | | | |
| the 12-well plate, and the | | | | | | | |
| action concentration of the | | | | | | | |
| siRNAs in each group was | | HPV16 siRNA | | | | | |
| 120 nM | | CRPE43# | | | CRPE45# | | |
| HPV18 | 44# | 1:2 | 1:1 | 2:1 | 1:2 | 1:1 | 2:1 |
| siRNA | 46# | 1:2 | 1:1 | 2:1 | 1:2 | 1:1 | 2:1 |

### Experimental Method:

A routine 4-hour cell transfection method (properly modified according to the Lipofectamine 2000 product operation manual);

Reverse Transcription (RT)-real-time quantitative (real-time) PCR technology.

Gene knockout experiments can be evaluated by detecting changes in mRNA in siRNA-treated cells, and RT-PCR was used to amplify RNA isolated from the corresponding cells. Selection of appropriate upstream and downstream primers is an initial step in evaluating target gene knockdown and selecting appropriate cell lines. The primer sequences used for RT-PCR analysis are:

The sequences of HPV 16 PCR primers are as follows:

HPV16-1:
16E6-1F (191-461):
(SEQ ID NO. 287)
GGAATCCATATGCTGTATGT
(PCR product length: 270 bp)
16E6-1B (191-461):
(SEQ ID NO. 288)
CTACGTGTTCTTGATGATCT
HPV16-2:
16E6-2F (278-448):
(SEQ ID NO. 289)
CAACATTAGAACAGCAATAC
(PCR product length: 170 bp)
16E6-2B (278-448):
(SEQ ID NO. 290)
ATGATCTGCAACAAGACATA
HPV16-E7-1:
16 E7-1F (21-43):
(SEQ ID NO. 291)
ATTGCATGAATATATGTTAGATT
(PCR product length: 250 bp)
16E7-1B (248-270):
(SEQ ID NO. 292)
CACAATTCCTAGTGTGCCCATTA

The sequences of HPV 18 PCR primers are as follows:

HPV18-1:
18E6-1F (65-84):
(SEQ ID NO. 293)
ACACTTCACTGCAAGACATA
(PCR product length: 196 bp)
18E6-1B: (241-260):
(SEQ ID NO. 294)
CCATACACAGAGTCTGAATA
HPV18-2:
18E6-2F (107-126):
(SEQ ID NO. 295)
AGACAGTATTGGAACTTACA
(PCR product length: 151 bp)
18E6-2B (238-257):
(SEQ ID NO. 296)
TACACAGAGTCTGAATAATG
HPV18-E7-1:
18 E7-1F (38-54):
(SEQ ID NO. 297)
TGCATTTAGAGCCCCAA
(PCR product length: 253 bp)
18E7-1B (275-291):
(SEQ ID NO. 298)
CACAAAGGACAGGGTGT

Total RNAs were extracted from cell culture or tumor tissue using the RNeasy mini kit (Qiagen, California) according to the manufacturer's instructions. For RT-PCR, the first cDNA strand was synthesized by using a cDNA synthesis kit (GE Healthcare, Chicago, IL) according to the manufacturer's instructions. The PCR reaction was started with lower cycle numbers, from 25, 30 to 35, to avoid the possible amplification plateau. Both Geneamp 9700 Thermalcycler and Taqman (ABI, CA) were used for PCR analysis. The amplicons were subjected to the gel electrophoresis analysis.

The PCR primer sequence expressed by E7 genes in HPV-16 in SiHa cell lines was as follows:

16E7-Forward:
(SEQ ID NO. 299)
ATTGCATGAATATATGTTAGATT
16E7-Reverse:
(SEQ ID NO. 300)
CACAATTCCTAGTGTGCCCATTA;

The PCR primer sequence expressed by E7 genes in HPV-18 in HeLa cell lines was as follows:

18E7-1Forward:
(SEQ ID NO. 301)
TGCATTTAGAGCCCCAA
18E7-1Reverse:
(SEQ ID NO. 302)
CACAAAGGACAGGGTGT

### Result Analysis:

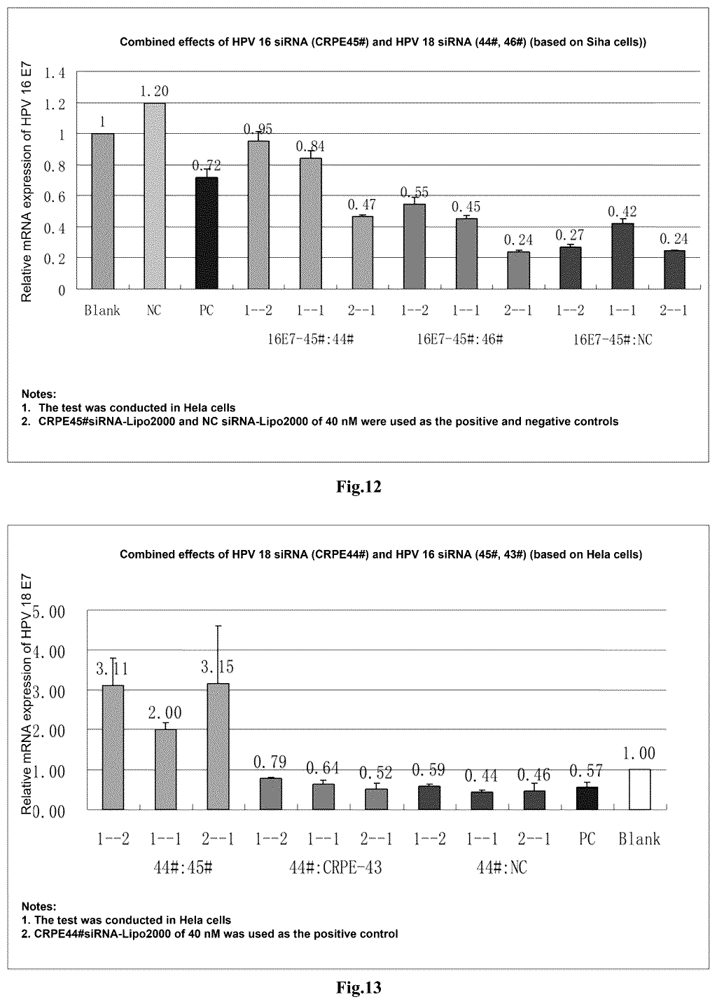

In the results of FIGS. 11-14, it can be preliminarily determined that the pairing effect (siha cells) of HPV 16 siRNA (CRPE43#) and HPV 18 siRNA (46#) is relatively good.

### Embodiment 6. Coupling of Anti-HPV siRNAs and Small Molecule Drugs

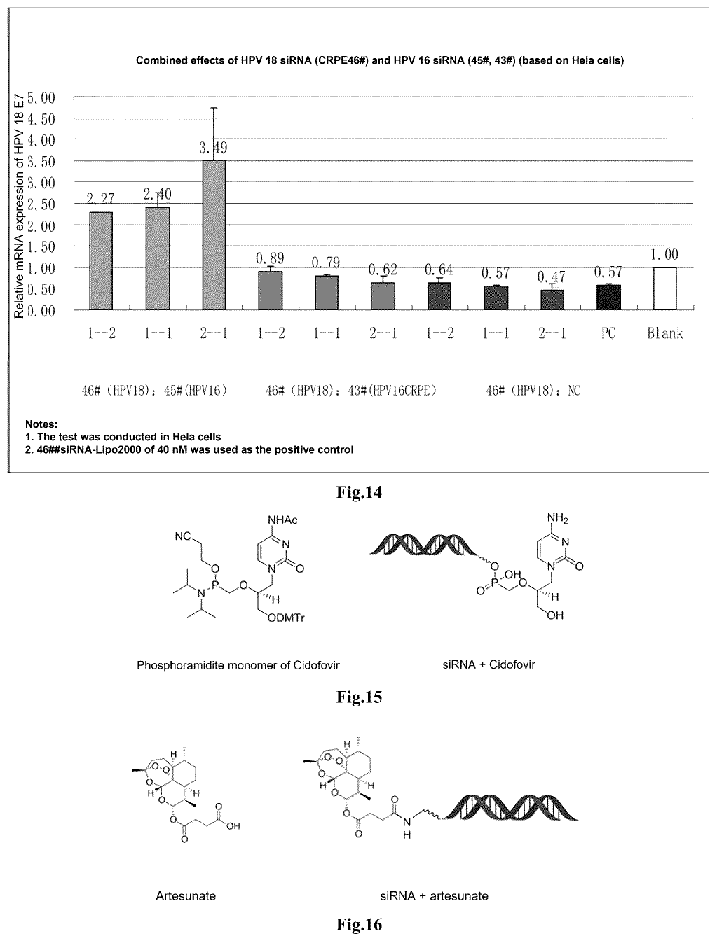

FIG. 15 shows the coupling mode and structure of siRNA molecules and small molecule drugs such as nucleotide analogues. Both cidofovir and brincidofovir are nucleotide analogues, and the molecules contain amino groups, hydroxyl groups, and phosphoric acid active groups. They can be molecularly modified by general nucleic acid chemistry professionals to make phosphoramidite monomers suitable for solid-phase synthesis. The phosphoramidite monomers obtained through such modification can be directly used in the solid-phase synthesis of the siRNAs, and one or more cidofovir or brincidofovir molecule(s) were inserted at any position of the siRNAs.

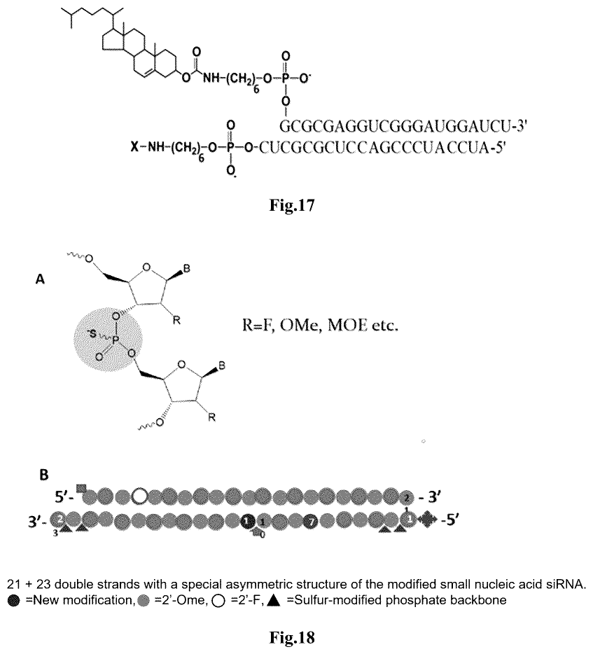

FIG. 16 shows the coupling mode and structure of siRNA molecules and artemisinin derivatives. Both the artesunate and the dihydroartemisinin are artemisinin derivatives, and their molecules contain carboxylic acid or hydroxyl active groups. They can be linked to the ends or side strands of siRNAs by common methods such as an addition reaction and a condensation reaction. In addition, through the phosphate groups, different molecules can be efficiently linked to one ends of the siRNAs (FIG. 17).

### Embodiment 7. Modification of siRNAs

siRNAs comprise special 2′-OMe, 2′-F, 2′-MOE, sulfur-modified phosphate backbones, base modifications, antisense and sense 5′ end modifications (FIG. 18A), and other chemically modified small nucleic acids to improve the stability of the small nucleic acid siRNAs and reduce the off-target effect and immune response of the small nucleic acid siRNAs.

The modified small nucleic acid siRNAs include 19+2 double strands, 21+23 double strands, etc. with a special asymmetric structure (FIG. 18B).

The present invention has been described in detail above, and its purpose is to allow those familiar with this field to understand the content of the present invention and implement it, but it cannot limit the scope of protection of the present invention. Effect variations or modifications should be covered within the protection scope of the present invention.

### REFERENCES

1. L Gissmann and H Z Hausen. Human papillomavirus DNA: physical mapping and genetic heterogeneity. Proc Natl Acad Sci USA., 1976. 73 (4): 1310-3.
2. U Bantel-Schaal L and H. zur Hausen. Characterization of the DNA of a defective human parvovirus isolated from a genital site. Virology. 1984. 134 (1): 52-63.
3. L Crawford, papillomaviruses and cervical tumors. Nature. 1984. 310 (5972): 16.
4. Gissmann L, et al., Presence of human papillomavirus in genital tumors. J Invest Dermatol. 1984. 83 (1 Suppl): 26s-28s.
5. Kahn J A HPV vaccination for the prevention of cervical intraepithelial neoplasia. N Engl J Med. 2009.

361 (3): 271-8.
6. Information from FDA and CDC on Gardasil and its Safety.
7. Trottier H and A N Burchell, Epidemiology of mucosal human papillomavirus infection and associated diseases. Public Health Genomics. 2009. 12 (5-6): 291-307.
8. Hellner, K., et al. HPV 16 E7 oncogene expression in normal human epithelial cells causes molecular changes indicative of an epithelial to mesenchymal transition. Virology. 2009 Aug. 15 [cited 391 1]; 2009 Jun. 26: [57-63]. Available from: http://www.ncbi.nlm.nih.gov/entrez/query.fcgi?cmd=Retrieve&db=PubMed&dopt-Citation&list_uids=19 552933.
9. Information from FDA and CDC on Gardasil and its Safety.
10. http://www.fda.gov/BiologicsBloodVaccines/Vaccines/ApprovedProducts/ucm172678.htm.
11. Kohout T, Stewart A. “New Report Examines Laws that Would Mandate HPV Vaccine for Young Women”. Jacobs Institute for Women's Health, George Washington University. http://www.jiwh.org/content.cfm?sectionid=167
12. “1 in 4 US teen girls got cervical cancer shot”. http://www.washingtonpost.com/wpdyn/content/article/2008/10/09/AR2008100901452.html?sub=new2.
13. Cervarix Marketing in Kenya
14. “Information from FDA and CDC on Gardasil and its Safety.”
15. PA Sharp RNA interference—2001. Genes Dev. 2001. 15 (5): 485-90.
16. Li L and Y Shen. Overcoming obstacles to develop effective and safe siRNA therapeutics. Expert Opin Biol Ther. 2009. 9 (5): 609-19.
17. http://www.clinicaltrials.gov/ct2/results?term=siRNA.
18. Li B J et al., Using siRNA in prophylactic and therapeutic regimens against SARS coronavirus in Rhesus macaque. Nat Med. 2005. 11 (9): 944-51.
19. F Y Xie, M C Woodle and P Y Lu. Harnessing in vivo siRNA delivery for drug discovery and therapeutic development. Drug Discov Today. 2006. 11 (1-2): 67-73.

1. A nucleic acid polypeptide nano-pharmaceutical composition, comprising:
siRNAs of HPV 16-E7 and HPV 18-E7, conjugates of the siRNAs and small molecule drugs, and a pharmaceutically acceptable carrier suitable for delivering the drugs in vivo, or a nano-drug consisting of the carrier and the siRNAs or the conjugate of the siRNAs and the small molecule drug,
wherein the composition can be used for treating and preventing related diseases caused by human papillomavirus infection through local administration or systemic administration.

2. The pharmaceutical composition of claim 1, wherein the nucleic acid components comprise at least one siRNA targeting HPV 16-E7 mRNA and HPV 18-E7 mRNA, or at least one mRNA as a tumor-specific antigen; the siRNA molecules comprise a sense strand and an antisense strand, the sequence of the sense strand is selected from any one of SEQ ID No. 1-143, and the antisense strand is selected from any one of SEQ ID No. 144-286, and is complementary to the sense strand.

3. The pharmaceutical composition of claim 2, wherein the siRNAs are sequences designed according to the mRNAs of cotton rabbit papillomaviruses and homologous sequences of HPV 16 E7; and/or
the siRNAs are sequences designed according to the cotton rabbit papillomaviruses and mRNA of HPV 18 E7.

4. The pharmaceutical composition of claim 1, wherein the siRNAs comprise a human HPV 16-CRPV-E7 siRNA-43 sequence with
a sense strand of 5′-GGAAGACCUGCUGAUGGGCACCCU-3′,
and an antisense strand of 5′-AGGGUGCCCAUCAGCAGGUCUUCC-3′; and/or
the siRNAs comprise an HPV 16-E7 siRNA-45# sequence with
a sense strand of 5′-GCACCCUGGGCAUCCUGUGCCCCAU-3′,
and an antisense strand of 5′-AUGGGGCACAGGAUGCCCAGGGUGC-3′; and/or
the siRNAs comprise an HPV 18-E7 siRNA-46# sequence with
a sense strand of 5′-GCUGUUUCUGAACACCCUGUCCUUU-3′,
and an antisense strand of 5′-AAAGGACAGGGUGUUCAGAAACAGC-3′; and/or
the siRNAs comprise an HPV 18-E7 siRNA-44# sequence with
a sense strand of 5′-GCUCAGCAGACGACCUUCGAGCAUU-3′,
and an antisense strand of 5′-AAUGCUCGAAGGUCGUCUGCUGAGC-3′.

5-8. (canceled)

9. The pharmaceutical composition of claim 1, wherein the siRNAs are double-stranded, and can be chemically modified to further optimize the targeting nature and inhibitory effect.

10. The pharmaceutical composition of claim 1, wherein
the siRNAs comprise HPV 16-CRPV-E7 siRNA-43# and HPV 18-E7 siRNA-44#, and the HPV 16-CRPV-E7 siRNA-43# and the HPV 18-E7 siRNA-44# are mixed into double-target siRNA inhibitors; and/or,
the siRNAs comprise HPV 16-CRPV-E7 siRNA-43# and HPV 18-E7 siRNA-46#, and the HPV 16-CRPV-E7 siRNA-43# and the HPV 18-E7 siRNA-46# are mixed into double-target siRNA inhibitors; and/or
the siRNAs comprise HPV 16-E7 siRNA-45# and HPV 18-E7 siRNA-44#, and the HPV 16-E7 siRNA-45# and the HPV 18-E7 siRNA-44# are mixed into double-target siRNA inhibitors; and/or
the siRNAs comprise HPV 16-E7 siRNA-45# and HPV 18-E7 siRNA-46#, and the HPV 16-E7 siRNA-45# and the HPV 18-E7 siRNA-46# are mixed into double-target siRNA inhibitors; and/or
the siRNAs comprise HPV 16-CRPV-E7 siRNA-43#, HPV 16-E7 siRNA-45# and HPV 18-E7 siRNA-44#, and the HPV 16-CRPV-E7 siRNA-43#, the HPV 16-E7 siRNA-45# and the HPV 18-E7 siRNA-44# are mixed into three-target siRNA inhibitors; and/or,
the siRNAs comprise HPV 16-CRPV-E7 siRNA-43#, HPV 16-E7 siRNA-45# and HPV 18-E7 siRNA-46#, and the HPV 16-CRPV-E7 siRNA-43#, the HPV 16-E7 siRNA-45# and the HPV 18-E7 siRNA-46# are mixed into three-target siRNA inhibitors; and/or,
the siRNAs comprise HPV 16-CRPV-E7 siRNA-43#, HPV 18-E7 siRNA-44# and HPV 18-E7 siRNA-46#, and the HPV 16-CRPV-E7 siRNA-43#, the HPV 18-E7 siRNA-44# and the HPV 18-E7 siRNA-46# are mixed into three-target siRNA inhibitors; and/or,
the siRNAs comprise HPV 16-E7 siRNA-45#, HPV 18-E7 siRNA-44# and HPV 18-E7 siRNA-46#, and the HPV 16-E7 siRNA-45#, the HPV 18-E7 siRNA-44# and the HPV 18-E7 siRNA-46# are mixed into three-target siRNA inhibitors.

11. (canceled)

12. A pharmaceutical composition for preventing or treating HPV infection, wherein the active ingredients of the pharmaceutical composition comprise siRNA molecules for inhibiting HPV replication and another molecule, the another molecule comprises one or more of siRNA molecule(s) for inhibiting human immune regulation related genes, anti-HPV small molecule compounds, a cervical cancer mRNA vaccine, or an anti-HPV monoclonal antibody.

13. The pharmaceutical composition of claim 12, wherein the siRNA molecules for inhibiting human immune regulation related genes are siRNA molecules for inhibiting immune checkpoints, and are selected from one or more of siRNA molecule(s) for inhibiting PD-1, siRNA molecules for inhibiting PD-L1, siRNA molecules for inhibiting LAG-3, siRNA molecules for inhibiting TIM-3, siRNA molecules for inhibiting VISTA, siRNA molecules for inhibiting TIGIT, or siRNA molecules for inhibiting CTLA-4/B7.

14. The pharmaceutical composition of claim 1, wherein the pharmaceutically acceptable carrier(s) are one or more of physiological saline, sugar solutions, polypeptides, polymers, lipids, cream gels, micellar materials, metal nanoparticles, dendrimers or HK polymers; and/or the N:P mass ratio of the carrier to the siRNAs is between 16:1 and 1:8.

15. The pharmaceutical composition of 14, wherein the pharmaceutically acceptable carrier is a polypeptide carrier, and the polypeptide carrier is a carrier material suitable for in vivo introduction, namely positively charged histidine-lysine co-polymers or modifiers thereof.

16. The pharmaceutical composition of claim 15, wherein the modifiers of the histidine-lysine co-polymers are branched histidine-lysine polymers with one branched histidine added to each branch; and/or
the histidine-lysine co-polymers adopt H3K4b or H3K(+H)4b.

17-18. (canceled)

19. The pharmaceutical composition of claim 1, wherein the composition comprises at least 2 siRNA molecules and a pharmaceutical carrier, and the siRNA molecules are combined with at least 2 mRNA molecules encoding part of human papillomavirus polypeptides or proteins; and/or the siRNA comprises 2 siRNA molecules in a ratio of 1:2 and 1:1 or 2:1.

20. (canceled)

21. The pharmaceutical composition of claim 1, wherein the carrier comprises a histidine-lysine polymer, the polymer and one, two or more siRNA molecule(s) form a nucleic acid polypeptide nano-pharmaceutical composition, and the diameter of the nano-drug is 50-300 nm.

22. A method for treating a mammal infected with HPV, comprising administering to a mammal a pharmaceutically effective dose of the composition of claim 1.

23. A method for treating a mammal infected with HPV and HIV and/or HSV, comprising administering to a mammal a pharmaceutically effective dose of the composition of claim 1.

24. A method for treating a mammal infected with HPV and fungi, comprising administering to the mammal a pharmaceutically effective dose of the composition of claim 1.

25. The pharmaceutical composition of claim 1, wherein a single siRNA is combined with an mRNA encoded by an HPV gene, wherein 20-40 nucleotide pairs of HPV 16 or HPV 18 are inserted into the same “reading frame” at the end of an E7 gene of the cotton tail rabbit papillomavirus to form a fusion protein, and the 20-40 nucleotide pairs can be used as the attack sequence sites of the siRNA; and/or
the siRNA can be subjected to specific chemical modifications;
the chemically modified small nucleic acids comprising special 2′-OMe, 2′-F, 2′-MOE, sulfur-modified phosphate backbones, base modification, antisense and sense 5′ end modifications increase the stability of siRNAs and reduce the off-target effects and immune response of the siRNAs.

26. (canceled)

27. The pharmaceutical composition of claim 26, wherein the modified siRNA comprises 19+2 double strands, and 21+23 double strands with a special asymmetric structure.

28. An siRNA-small molecule drug conjugate, wherein the siRNA-small molecule drug conjugate is formed by covalent bond coupling of the siRNA molecule for inhibiting HPV viruses and a small molecule compound for inhibiting HPV viruses.

29. The siRNA-small molecule drug conjugate of claim 28, wherein the small molecule compound for inhibiting HPV viruses is a nucleotide analogue and/or artemisinin derivative for inhibiting the HPV viruses; and/or
the nucleotide analogues for inhibiting HPV viruses are selected from one or more of cidofovir and brincidofovir; and/or
the artemisinin derivative is selected from one or more of artesunate and dihydroartemisinin derivatives; and/or
the application of the siRNA-small molecule drug conjugate in the prevention or treatment of HPV-induced cervical precancerous lesions, skin lesions, condyloma acuminata, and other diseases.

30-32. (canceled)


SEQUENCE LISTING (303 sequences)

SEQ ID NO: 1 - RNA, 25 nt, Artificial Sequence
  the sequence is synthesized
gcauggaccuaaggcaacauugcaa

SEQ ID NO: 2 - RNA, 25 nt, Artificial Sequence
  the sequence is synthesized
gguugaccuucuaugucacgagcaa

SEQ ID NO: 3 - RNA, 25 nt, Artificial Sequence
  the sequence is synthesized
gcaauuaagcgacucagaggaagaa

SEQ ID NO: 4 - RNA, 25 nt, Artificial Sequence
  the sequence is synthesized
cgaugaaauagauggaguuaaucau

SEQ ID NO: 5 - RNA, 25 nt, Artificial Sequence
  the sequence is synthesized
cgagccgaaccacaacgucacacaa

SEQ ID NO: 6 - RNA, 25 nt, Artificial Sequence
  the sequence is synthesized
gccagaauugagcuaguaguagaaa

SEQ ID NO: 7 - RNA, 25 nt, Artificial Sequence
  the sequence is synthesized
gcucagcagacgaccuucgagcauu

SEQ ID NO: 8 - RNA, 25 nt, Artificial Sequence
  the sequence is synthesized
gcuguuucugaacacccuguccuuu

SEQ ID NO: 9 - RNA, 25 nt, Artificial Sequence
  the sequence is synthesized
gcauggagauacaccuacauugcau

SEQ ID NO: 10 - RNA, 25 nt, Artificial Sequence
  the sequence is synthesized
ggagauacaccuacauugcaugaau

SEQ ID NO: 11 - RNA, 25 nt, Artificial Sequence
  the sequence is synthesized
gcaugaauauauguuagauuugcaa

SEQ ID NO: 12 - RNA, 25 nt, Artificial Sequence
  the sequence is synthesized
ggacagagcccauuacaauauugua

SEQ ID NO: 13 - RNA, 25 nt, Artificial Sequence
  the sequence is synthesized
gcccauuacaauauuguaaccuuuu

SEQ ID NO: 14 - RNA, 25 nt, Artificial Sequence
  the sequence is synthesized
gcaagugugacucuacgcuucgguu

SEQ ID NO: 15 - RNA, 25 nt, Artificial Sequence
  the sequence is synthesized
gcguacaaagcacacacguagacau

SEQ ID NO: 16 - RNA, 25 nt, Artificial Sequence
  the sequence is synthesized
cguacaaagcacacacguagacauu

SEQ ID NO: 17 - RNA, 25 nt, Artificial Sequence
  the sequence is synthesized
gcacacacguagacauucguacuuu

SEQ ID NO: 18 - RNA, 25 nt, Artificial Sequence
  the sequence is synthesized
ggaagaccuguuaaugggcacacua

SEQ ID NO: 19 - RNA, 25 nt, Artificial Sequence
  the sequence is synthesized
ccuguuaaugggcacacuaggaauu

SEQ ID NO: 20 - RNA, 25 nt, Artificial Sequence
  the sequence is synthesized
gcacacuaggaauugugugccccau

SEQ ID NO: 21 - RNA, 24 nt, artificial sequence
  the sequence is synthesized
gcaugaauauauguuggaucugca

SEQ ID NO: 22 - RNA, 24 nt, Artificial Sequence
  the sequence is synthesized
ggacagagcccacuacaacaucgu

SEQ ID NO: 23 - RNA, 25 nt, Artificial Sequence
  the sequence is synthesized
gcccacuacaacaucgugaccuuuu

SEQ ID NO: 24 - RNA, 24 nt, Artificial Sequence
  the sequence is synthesized
ggaagaccugcugaugggcacccu

SEQ ID NO: 25 - RNA, 24 nt, Artificial Sequence
  the sequence is synthesized
ccugcugaugggcacccugggcau

SEQ ID NO: 26 - RNA, 25 nt, Artificial Sequence
  the sequence is synthesized
gcacccugggcauccugugccccau

SEQ ID NO: 27 - RNA, 25 nt, Artificial Sequence
  the sequence is synthesized
cccacaggagcgacccagaaaguua

SEQ ID NO: 28 - RNA, 25 nt, Artificial Sequence
  the sequence is synthesized
cgacccagaaaguuaccacaguuau

SEQ ID NO: 29 - RNA, 25 nt, Artificial Sequence
  the sequence is synthesized
ccacaguuaugcacagagcugcaaa

SEQ ID NO: 30 - RNA, 25 nt, Artificial Sequence
  the sequence is synthesized
cgacgugagguauaugacuuugcuu

SEQ ID NO: 31 - RNA, 25 nt, Artificial Sequence
  the sequence is synthesized
gggaauccauaugcuguaugugaua

SEQ ID NO: 32 - RNA, 25 nt, Artificial Sequence
  the sequence is synthesized
gcaauacaacaaaccguugugugau

SEQ ID NO: 33 - RNA, 25 nt, Artificial Sequence
  the sequence is synthesized
cgguggaccggucgauguaugucuu

SEQ ID NO: 34 - RNA, 25 nt, Artificial Sequence
  the sequence is synthesized
cgauguaugucuuguugcagaucau

SEQ ID NO: 35 - RNA, 25 nt, Artificial Sequence
  the sequence is synthesized
ggguacgggauguaauggaugguuu

SEQ ID NO: 36 - RNA, 25 nt, Artificial Sequence
  the sequence is synthesized
cggguauggcaauacugaaguggaa

SEQ ID NO: 37 - RNA, 25 nt, Artificial Sequence
  the sequence is synthesized
gcugcauuuggacuuacacccagua

SEQ ID NO: 38 - RNA, 25 nt, Artificial Sequence
  the sequence is synthesized
ggagacacgccagaauggauacaaa

SEQ ID NO: 39 - RNA, 25 nt, Artificial Sequence
  the sequence is synthesized
ggauugugcaacaauguguagacau

SEQ ID NO: 40 - RNA, 25 nt, Artificial Sequence
  the sequence is synthesized
ggugcagcuaacacagguaaaucau

SEQ ID NO: 41 - RNA, 25 nt, Artificial Sequence
  the sequence is synthesized
ggauguaaagcauagaccauuggua

SEQ ID NO: 42 - RNA, 25 nt, Artificial Sequence
  the sequence is synthesized
cgauggagacucuuugccaacguuu

SEQ ID NO: 43 - RNA, 25 nt, Artificial Sequence
  the sequence is synthesized
ggagacucuuugccaacguuuaaau

SEQ ID NO: 44 - RNA, 25 nt, Artificial Sequence
  the sequence is synthesized
gcuauuuauuacaaggccagagaaa

SEQ ID NO: 45 - RNA, 25 nt, Artificial Sequence
  the sequence is synthesized
ggaagugcaguuugauggagacaua

SEQ ID NO: 46 - RNA, 25 nt, Artificial Sequence
  the sequence is synthesized
gggucaaguugacuauuaugguuua

SEQ ID NO: 47 - RNA, 25 nt, Artificial Sequence
  the sequence is synthesized
ggaaguucaugcggguggucaggua

SEQ ID NO: 48 - RNA, 25 nt, Artificial Sequence
  the sequence is synthesized
cgacccauaccaaagccgucgccuu

SEQ ID NO: 49 - RNA, 25 nt, Artificial Sequence
  the sequence is synthesized
ccaagaucagagccagacaccggaa

SEQ ID NO: 50 - RNA, 25 nt, Artificial Sequence
  the sequence is synthesized
ggcauuggacaggacauaauguaaa

SEQ ID NO: 51 - RNA, 25 nt, Artificial Sequence
  the sequence is synthesized
gcaacgaaguauccucuccugaaau

SEQ ID NO: 52 - RNA, 25 nt, Artificial Sequence
  the sequence is synthesized
cgaaguauccucuccugaaauuauu

SEQ ID NO: 53 - RNA, 25 nt, Artificial Sequence
  the sequence is synthesized
cgacccauaccaaagccgucgccuu

SEQ ID NO: 54 - RNA, 25 nt, Artificial Sequence
  the sequence is synthesized
gccgucgccuugggcaccgaagaaa

SEQ ID NO: 55 - RNA, 25 nt, Artificial Sequence
  the sequence is synthesized
ggcaccgaagaaacacagacgacua

SEQ ID NO: 56 - RNA, 25 nt, Artificial Sequence
  the sequence is synthesized
gcaccgaagaaacacagacgacuau

SEQ ID NO: 57 - RNA, 25 nt, Artificial Sequence
  the sequence is synthesized
ccaagaucagagccagacaccggaa

SEQ ID NO: 58 - RNA, 25 nt, Artificial Sequence
  the sequence is synthesized
cgugcaucggcuacccaacuuuaua

SEQ ID NO: 59 - RNA, 25 nt, Artificial Sequence
  the sequence is synthesized
ggguacaggcggacgcacuggguau

SEQ ID NO: 60 - RNA, 25 nt, Artificial Sequence
  the sequence is synthesized
cccagauguaucaggauuuaguauu

SEQ ID NO: 61 - RNA, 25 nt, Artificial Sequence
  the sequence is synthesized
cgcccaguggcacgccuaggauuau

SEQ ID NO: 62 - RNA, 25 nt, Artificial Sequence
  the sequence is synthesized
ccacucccacuaaacuuauuacaua

SEQ ID NO: 63 - RNA, 25 nt, Artificial Sequence
  the sequence is synthesized
gcagccucaccuacuucuauuaaua

SEQ ID NO: 64 - RNA, 25 nt, Artificial Sequence
  the sequence is synthesized
ccagggucuccacaauauacaauua

SEQ ID NO: 65 - RNA, 25 nt, Artificial Sequence
  the sequence is synthesized
ggcugccuagugaggccacugucua

SEQ ID NO: 66 - RNA, 25 nt, Artificial Sequence
  the sequence is synthesized
gcugguuugggccuguguagguguu

SEQ ID NO: 67 - RNA, 25 nt, Artificial Sequence
  the sequence is synthesized
gcagcaaaugcagguguggauaaua

SEQ ID NO: 68 - RNA, 25 nt, Artificial Sequence
  the sequence is synthesized
cccauguaccaauguugcaguaaau

SEQ ID NO: 69 - RNA, 25 nt, Artificial Sequence
  the sequence is synthesized
gggucuacugcaaauuuagccaguu

SEQ ID NO: 70 - RNA, 25 nt, Artificial Sequence
  the sequence is synthesized
ggaggcacacuagaagauacuuaua

SEQ ID NO: 71 - RNA, 25 nt, Artificial Sequence
  the sequence is synthesized
ccucaucuaccucuacaacugcuaa

SEQ ID NO: 72 - RNA, 25 nt, Artificial Sequence
  the sequence is synthesized
gcaagacauagaaauaaccugugua

SEQ ID NO: 73 - RNA, 25 nt, Artificial Sequence
  the sequence is synthesized
ccuguguauauugcaagacaguauu

SEQ ID NO: 74 - RNA, 25 nt, Artificial Sequence
  the sequence is synthesized
cccaugcugcaugccauaaauguau

SEQ ID NO: 75 - RNA, 25 nt, Artificial Sequence
  the sequence is synthesized
ggugccugcggugccagaaaccguu

SEQ ID NO: 76 - RNA, 25 nt, Artificial Sequence
  the sequence is synthesized
ccagaaaccguugaauccagcagaa

SEQ ID NO: 77 - RNA, 25 nt, Artificial Sequence
  the sequence is synthesized
gggcacuauagaggccagugccauu

SEQ ID NO: 78 - RNA, 25 nt, Artificial Sequence
  the sequence is synthesized
ccgagcacgacaggaacgacuccaa

SEQ ID NO: 79 - RNA, 25 nt, Artificial Sequence
  the sequence is synthesized
ggaacgacuccaacgacgcagagaa

SEQ ID NO: 80 - RNA, 25 nt, Artificial Sequence
  the sequence is synthesized
gggcacggguuguaacggcugguuu

SEQ ID NO: 81 - RNA, 25 nt, Artificial Sequence
  the sequence is synthesized
ggcaauguauguaguggcggcagua

SEQ ID NO: 82 - RNA, 25 nt, Artificial Sequence
  the sequence is synthesized
ggguuacagcuauauuuggaguaaa

SEQ ID NO: 83 - RNA, 25 nt, Artificial Sequence
  the sequence is synthesized
gcuauauuuggaguaaacccaacaa

SEQ ID NO: 84 - RNA, 25 nt, Artificial Sequence
  the sequence is synthesized
ccuuauuagcagacagcaacagcaa

SEQ ID NO: 85 - RNA, 25 nt, Artificial Sequence
  the sequence is synthesized
cguguuggacauacuuugauaccua

SEQ ID NO: 86 - RNA, 25 nt, Artificial Sequence
  the sequence is synthesized
ggaagaggaagaugcagacaccgaa

SEQ ID NO: 87 - RNA, 25 nt, Artificial Sequence
  the sequence is synthesized
cgaaggaaacccuuucggaacguuu

SEQ ID NO: 88 - RNA, 25 nt, Artificial Sequence
  the sequence is synthesized
gcaagggaacauggcauacagacau

SEQ ID NO: 89 - RNA, 25 nt, Artificial Sequence
  the sequence is synthesized
ggaauacagaaccuacucacugcuu

SEQ ID NO: 90 - RNA, 25 nt, Artificial Sequence
  the sequence is synthesized
ggacaguguguauuauaugacugau

SEQ ID NO: 91 - RNA, 25 nt, Artificial Sequence
  the sequence is synthesized
cgguauccgcuacucagcuuguuaa

SEQ ID NO: 92 - RNA, 25 nt, Artificial Sequence
  the sequence is synthesized
gguaacacuacgccuauaauacauu

SEQ ID NO: 93 - RNA, 25 nt, Artificial Sequence
  the sequence is synthesized
ggaauacugacuguaacauaccaua

SEQ ID NO: 94 - RNA, 25 nt, Artificial Sequence
  the sequence is synthesized
cgacacgguauccgcuacucagcuu

SEQ ID NO: 95 - RNA, 25 nt, Artificial Sequence
  the sequence is synthesized
gguauccgcuacucagcuuguuaaa

SEQ ID NO: 96 - RNA, 25 nt, Artificial Sequence
  the sequence is synthesized
cgcuacucagcuuguuaaacagcua

SEQ ID NO: 97 - RNA, 25 nt, Artificial Sequence
  the sequence is synthesized
gcauuguggaccugucaacccacuu

SEQ ID NO: 98 - RNA, 25 nt, Artificial Sequence
  the sequence is synthesized
ccacuucucggugcagcuacaccua

SEQ ID NO: 99 - RNA, 25 nt, Artificial Sequence
  the sequence is synthesized
cggaaacucuguagugguaacacua

SEQ ID NO: 100 - RNA, 25 nt, Artificial Sequence
  the sequence is synthesized
gccaucugucuguaugugugcguau

SEQ ID NO: 101 - RNA, 25 nt, Artificial Sequence
  the sequence is synthesized
gcauggguauugguauuuguguaua

SEQ ID NO: 102 - RNA, 25 nt, Artificial Sequence
  the sequence is synthesized
cccugccacagcauucacaguauau

SEQ ID NO: 103 - RNA, 25 nt, Artificial Sequence
  the sequence is synthesized
gccacagcauucacaguauauguau

SEQ ID NO: 104 - RNA, 25 nt, Artificial Sequence
  the sequence is synthesized
ccacagcauucacaguauauguauu

SEQ ID NO: 105 - RNA, 25 nt, Artificial Sequence
  the sequence is synthesized
gcccauguuacuauugcauauacau

SEQ ID NO: 106 - RNA, 25 nt, Artificial Sequence
  the sequence is synthesized
gcaaacgggcuucgguaacugacuu

SEQ ID NO: 107 - RNA, 25 nt, artificial sequence
  the sequence is synthesized
ggguacauuccauugggugggcguu

SEQ ID NO: 108 - RNA, 25 nt, Artificial Sequence
  the sequence is synthesized
ggguuugauauaacaucugcgggua

SEQ ID NO: 109 - RNA, 25 nt, Artificial Sequence
  the sequence is synthesized
cccuacaucuggaacacauggguau

SEQ ID NO: 110 - RNA, 25 nt, Artificial Sequence
  the sequence is synthesized
ccuaccaacaagugucaguggcuaa

SEQ ID NO: 111 - RNA, 25 nt, Artificial Sequence
  the sequence is synthesized
gcaacuauguuuacccgcagcggua

SEQ ID NO: 112 - RNA, 25 nt, Artificial Sequence
  the sequence is synthesized
cggaggacaaugacuuguuugauau

SEQ ID NO: 113 - RNA, 25 nt, Artificial Sequence
  the sequence is synthesized
ccuccucuugggaugugccuguaua

SEQ ID NO: 114 - RNA, 25 nt, Artificial Sequence
  the sequence is synthesized
ccugccucuacacaguauauuggua

SEQ ID NO: 115 - RNA, 25 nt, Artificial Sequence
  the sequence is synthesized
gggugcaguuaccugacccaaauaa

SEQ ID NO: 116 - RNA, 25 nt, Artificial Sequence
  the sequence is synthesized
ggauauggugccauggacuuuagua

SEQ ID NO: 117 - RNA, 25 nt, Artificial Sequence
  the sequence is synthesized
ccucugacucccaguuguuuaauaa

SEQ ID NO: 118 - RNA, 25 nt, Artificial Sequence
  the sequence is synthesized
gguagauaccacucccaguaccaau

SEQ ID NO: 119 - RNA, 25 nt, Artificial Sequence
  the sequence is synthesized
ccacucccaguaccaauuuaacaau

SEQ ID NO: 120 - RNA, 25 nt, Artificial Sequence
  the sequence is synthesized
ccaacuacuaguuugguggauacau

SEQ ID NO: 121 - RNA, 25 nt, Artificial Sequence
  the sequence is synthesized
ccacuacgucuucuaaaccugccaa

SEQ ID NO: 122 - RNA, 25 nt, Artificial Sequence
  the sequence is synthesized
ccuguugcuguggaugugacagcaa

SEQ ID NO: 123 - RNA, 25 nt, Artificial Sequence
  the sequence is synthesized
ggacaguggauauggcuauucugaa

SEQ ID NO: 124 - RNA, 25 nt, Artificial Sequence
  the sequence is synthesized
cgaggaagauggaagcaauagccaa

SEQ ID NO: 125 - RNA, 25 nt, Artificial Sequence
  the sequence is synthesized
ggaagcaauagccaagcguuuagau

SEQ ID NO: 126 - RNA, 25 nt, Artificial Sequence
  the sequence is synthesized
ggaaguauguuauggcagcacaguu

SEQ ID NO: 127 - RNA, 25 nt, Artificial Sequence
  the sequence is synthesized
cccuuuaguccuguaacuccugcuu

SEQ ID NO: 128 - RNA, 25 nt, Artificial Sequence
  the sequence is synthesized
ccuuuaguccuguaacuccugcuuu

SEQ ID NO: 129 - RNA, 25 nt, Artificial Sequence
  the sequence is synthesized
ccugcuuuaccuacaggcccuguuu

SEQ ID NO: 130 - RNA, 25 nt, Artificial Sequence
  the sequence is synthesized
ggcggccuagcgacagcacaguaua

SEQ ID NO: 131 - RNA, 25 nt, Artificial Sequence
  the sequence is synthesized
gcggccuagcgacagcacaguauau

SEQ ID NO: 132 - RNA, 25 nt, Artificial Sequence
  the sequence is synthesized
gcaugaauauauguuagauuugcaa

SEQ ID NO: 133 - RNA, 24 nt, Artificial Sequence
  the sequence is synthesized
gcaugaauauauguuggaucugca

SEQ ID NO: 134 - RNA, 25 nt, Artificial Sequence
  the sequence is synthesized
ggacagagcccauuacaauauugua

SEQ ID NO: 135 - RNA, 24 nt, Artificial Sequence
  the sequence is synthesized
ggacagagcccacuacaacaucgu

SEQ ID NO: 136 - RNA, 25 nt, Artificial Sequence
  the sequence is synthesized
gcccauuacaauaccguaaccuuuu

SEQ ID NO: 137 - RNA, 25 nt, Artificial Sequence
  the sequence is synthesized
gcccacuacaacaucgugaccuuuu

SEQ ID NO: 138 - RNA, 25 nt, Artificial Sequence
  the sequence is synthesized
ggaagaccuguuaaugggcacacua

SEQ ID NO: 139 - RNA, 24 nt, Artificial Sequence
  the sequence is synthesized
ggaagaccugcugaugggcacccu

SEQ ID NO: 140 - RNA, 25 nt, Artificial Sequence
  the sequence is synthesized
ccuguuaaugggcacacuaggaauu

SEQ ID NO: 141 - RNA, 24 nt, Artificial Sequence
  the sequence is synthesized
ccugcugaugggcacccugggcau

SEQ ID NO: 142 - RNA, 25 nt, Artificial Sequence
  the sequence is synthesized
gcacacuaggaauugugugccccau

SEQ ID NO: 143 - RNA, 25 nt, Artificial Sequence
  the sequence is synthesized
gcacccugggcauccugugccccau

SEQ ID NO: 144 - RNA, 25 nt, Artificial Sequence
  the sequence is synthesized
uugcaauguugccuuagguccaugc

SEQ ID NO: 145 - RNA, 25 nt, Artificial Sequence
  the sequence is synthesized
uugcucgugacauagaaggucaacc

SEQ ID NO: 146 - RNA, 25 nt, Artificial Sequence
  the sequence is synthesized
uucuuccucugagucgcuuaauugc

SEQ ID NO: 147 - RNA, 25 nt, Artificial Sequence
  the sequence is synthesized
augauuaacuccaucuauuucaucg

SEQ ID NO: 148 - RNA, 25 nt, Artificial Sequence
  the sequence is synthesized
uugugugacguugugguucggcucg

SEQ ID NO: 149 - RNA, 25 nt, Artificial Sequence
  the sequence is synthesized
uuucuacuacuagcucaauucuggc

SEQ ID NO: 150 - RNA, 25 nt, Artificial Sequence
  the sequence is synthesized
aaugcucgaaggucgucugcugagc

SEQ ID NO: 151 - RNA, 25 nt, Artificial Sequence
  the sequence is synthesized
aaaggacaggguguucagaaacagc

SEQ ID NO: 152 - RNA, 25 nt, Artificial Sequence
  the sequence is synthesized
augcaauguagguguaucuccaugc

SEQ ID NO: 153 - RNA, 25 nt, Artificial Sequence
  the sequence is synthesized
auucaugcaauguagguguaucucc

SEQ ID NO: 154 - RNA, 25 nt, Artificial Sequence
  the sequence is synthesized
uugcaaaucuaacauauauucaugc

SEQ ID NO: 155 - RNA, 25 nt, Artificial Sequence
  the sequence is synthesized
uacaauauuguaaugggcucugucc

SEQ ID NO: 156 - RNA, 25 nt, Artificial Sequence
  the sequence is synthesized
aaaagguuacaauauuguaaugggc

SEQ ID NO: 157 - RNA, 25 nt, Artificial Sequence
  the sequence is synthesized
aaccgaagcguagagucacacuugc

SEQ ID NO: 158 - RNA, 25 nt, Artificial Sequence
  the sequence is synthesized
augucuacgugugugcuuuguacgc

SEQ ID NO: 159 - RNA, 25 nt, Artificial Sequence
  the sequence is synthesized
aaugucuacgugugugcuuuguacg

SEQ ID NO: 160 - RNA, 25 nt, Artificial Sequence
  the sequence is synthesized
aaaguacgaaugucuacgugugugc

SEQ ID NO: 161 - RNA, 25 nt, Artificial Sequence
  the sequence is synthesized
uagugugcccauuaacaggucuucc

SEQ ID NO: 162 - RNA, 25 nt, Artificial Sequence
  the sequence is synthesized
aauuccuagugugcccauuaacagg

SEQ ID NO: 163 - RNA, 25 nt, Artificial Sequence
  the sequence is synthesized
auggggcacacaauuccuagugugc

SEQ ID NO: 164 - RNA, 24 nt, Artificial Sequence
  the sequence is synthesized
ugcagauccaacauauauucaugc

SEQ ID NO: 165 - RNA, 24 nt, Artificial Sequence
  the sequence is synthesized
acgauguuguagugggcucugucc

SEQ ID NO: 166 - RNA, 25 nt, Artificial Sequence
  the sequence is synthesized
aaaaggucacgauguuguagugggc

SEQ ID NO: 167 - RNA, 24 nt, Artificial Sequence
  the sequence is synthesized
agggugcccaucagcaggucuucc

SEQ ID NO: 168 - RNA, 24 nt, Artificial Sequence
  the sequence is synthesized
augcccagggugcccaucagcagg

SEQ ID NO: 169 - RNA, 25 nt, Artificial Sequence
  the sequence is synthesized
auggggcacaggaugcccagggugc

SEQ ID NO: 170 - RNA, 25 nt, Artificial Sequence
  the sequence is synthesized
uaacuuucugggucgcuccuguggg

SEQ ID NO: 171 - RNA, 25 nt, Artificial Sequence
  the sequence is synthesized
auaacugugguaacuuucugggucg

SEQ ID NO: 172 - RNA, 25 nt, Artificial Sequence
  the sequence is synthesized
uuugcagcucugugcauaacugugg

SEQ ID NO: 173 - RNA, 25 nt, Artificial Sequence
  the sequence is synthesized
aagcaaagucauauaccucacgucg

SEQ ID NO: 174 - RNA, 25 nt, Artificial Sequence
  the sequence is synthesized
uaucacauacagcauauggauuccc

SEQ ID NO: 175 - RNA, 25 nt, Artificial Sequence
  the sequence is synthesized
aucacacaacgguuuguuguauugc

SEQ ID NO: 176 - RNA, 25 nt, Artificial Sequence
  the sequence is synthesized
aagacauacaucgaccgguccaccg

SEQ ID NO: 177 - RNA, 25 nt, Artificial Sequence
  the sequence is synthesized
augaucugcaacaagacauacaucg

SEQ ID NO: 178 - RNA, 25 nt, Artificial Sequence
  the sequence is synthesized
aaaccauccauuacaucccguaccc

SEQ ID NO: 179 - RNA, 25 nt, Artificial Sequence
  the sequence is synthesized
uuccacuucaguauugccauacccg

SEQ ID NO: 180 - RNA, 25 nt, Artificial Sequence
  the sequence is synthesized
uacuggguguaaguccaaaugcagc

SEQ ID NO: 181 - RNA, 25 nt, Artificial Sequence
  the sequence is synthesized
uuuguauccauucuggcgugucucc

SEQ ID NO: 182 - RNA, 25 nt, Artificial Sequence
  the sequence is synthesized
augucuacacauuguugcacaaucc

SEQ ID NO: 183 - RNA, 25 nt, Artificial Sequence
  the sequence is synthesized
augauuuaccuguguuagcugcacc

SEQ ID NO: 184 - RNA, 25 nt, Artificial Sequence
  the sequence is synthesized
uaccaauggucuaugcuuuacaucc

SEQ ID NO: 185 - RNA, 25 nt, Artificial Sequence
  the sequence is synthesized
aaacguuggcaaagagucuccaucg

SEQ ID NO: 186 - RNA, 25 nt, Artificial Sequence
  the sequence is synthesized
auuuaaacguuggcaaagagucucc

SEQ ID NO: 187 - RNA, 25 nt, Artificial Sequence
  the sequence is synthesized
uuucucuggccuuguaauaaauagc

SEQ ID NO: 188 - RNA, 25 nt, Artificial Sequence
  the sequence is synthesized
uaugucuccaucaaacugcacuucc

SEQ ID NO: 189 - RNA, 25 nt, Artificial Sequence
  the sequence is synthesized
uaaaccauaauagucaacuugaccc

SEQ ID NO: 190 - RNA, 25 nt, Artificial Sequence
  the sequence is synthesized
uaccugaccacccgcaugaacuucc

SEQ ID NO: 191 - RNA, 25 nt, Artificial Sequence
  the sequence is synthesized
aaggcgacggcuuugguaugggucg

SEQ ID NO: 192 - RNA, 25 nt, Artificial Sequence
  the sequence is synthesized
uuccggugucuggcucugaucuugg

SEQ ID NO: 193 - RNA, 25 nt, Artificial Sequence
  the sequence is synthesized
uuuacauuauguccuguccaaugcc

SEQ ID NO: 194 - RNA, 25 nt, Artificial Sequence
  the sequence is synthesized
auuucaggagaggauacuucguugc

SEQ ID NO: 195 - RNA, 25 nt, Artificial Sequence
  the sequence is synthesized
aauaauuucaggagaggauacuucg

SEQ ID NO: 196 - RNA, 25 nt, Artificial Sequence
  the sequence is synthesized
aaggcgacggcuuugguaugggucg

SEQ ID NO: 197 - RNA, 25 nt, Artificial Sequence
  the sequence is synthesized
uuucuucggugcccaaggcgacggc

SEQ ID NO: 198 - RNA, 25 nt, Artificial Sequence
  the sequence is synthesized
uagucgucuguguuucuucggugcc

SEQ ID NO: 199 - RNA, 25 nt, Artificial Sequence
  the sequence is synthesized
auagucgucuguguuucuucggugc

SEQ ID NO: 200 - RNA, 25 nt, Artificial Sequence
  the sequence is synthesized
uuccggugucuggcucugaucuugg

SEQ ID NO: 201 - RNA, 25 nt, Artificial Sequence
  the sequence is synthesized
uauaaaguuggguagccgaugcacg

SEQ ID NO: 202 - RNA, 25 nt, Artificial Sequence
  the sequence is synthesized
auacccagugcguccgccuguaccc

SEQ ID NO: 203 - RNA, 25 nt, Artificial Sequence
  the sequence is synthesized
aauacuaaauccugauacaucuggg

SEQ ID NO: 204 - RNA, 25 nt, Artificial Sequence
  the sequence is synthesized
auaauccuaggcgugccacugggcg

SEQ ID NO: 205 - RNA, 25 nt, Artificial Sequence
  the sequence is synthesized
uauguaauaaguuuagugggagugg

SEQ ID NO: 206 - RNA, 25 nt, Artificial Sequence
  the sequence is synthesized
uauuaauagaaguaggugaggcugc

SEQ ID NO: 207 - RNA, 25 nt, Artificial Sequence
  the sequence is synthesized
uaauuguauauuguggagacccugg

SEQ ID NO: 208 - RNA, 25 nt, Artificial Sequence
  the sequence is synthesized
uagacaguggccucacuaggcagcc

SEQ ID NO: 209 - RNA, 25 nt, Artificial Sequence
  the sequence is synthesized
aacaccuacacaggcccaaaccagc

SEQ ID NO: 210 - RNA, 25 nt, Artificial Sequence
  the sequence is synthesized
uauuauccacaccugcauuugcugc

SEQ ID NO: 211 - RNA, 25 nt, Artificial Sequence
  the sequence is synthesized
auuuacugcaacauugguacauggg

SEQ ID NO: 212 - RNA, 25 nt, Artificial Sequence
  the sequence is synthesized
aacuggcuaaauuugcaguagaccc

SEQ ID NO: 213 - RNA, 25 nt, Artificial Sequence
  the sequence is synthesized
uauaaguaucuucuagugugccucc

SEQ ID NO: 214 - RNA, 25 nt, Artificial Sequence
  the sequence is synthesized
uuagcaguuguagagguagaugagg

SEQ ID NO: 215 - RNA, 25 nt, Artificial Sequence
  the sequence is synthesized
uacacagguuauuucuaugucuugc

SEQ ID NO: 216 - RNA, 25 nt, Artificial Sequence
  the sequence is synthesized
aauacugucuugcaauauacacagg

SEQ ID NO: 217 - RNA, 25 nt, Artificial Sequence
  the sequence is synthesized
auacauuuauggcaugcagcauggg

SEQ ID NO: 218 - RNA, 25 nt, Artificial Sequence
  the sequence is synthesized
aacgguuucuggcaccgcaggcacc

SEQ ID NO: 219 - RNA, 25 nt, Artificial Sequence
  the sequence is synthesized
uucugcuggauucaacgguuucugg

SEQ ID NO: 220 - RNA, 25 nt, Artificial Sequence
  the sequence is synthesized
aauggcacuggccucuauagugccc

SEQ ID NO: 221 - RNA, 25 nt, Artificial Sequence
  the sequence is synthesized
uuggagucguuccugucgugcucgg

SEQ ID NO: 222 - RNA, 25 nt, Artificial Sequence
  the sequence is synthesized
uucucugcgucguuggagucguucc

SEQ ID NO: 223 - RNA, 25 nt, Artificial Sequence
  the sequence is synthesized
aaaccagccguuacaacccgugccc

SEQ ID NO: 224 - RNA, 25 nt, Artificial Sequence
  the sequence is synthesized
uacugccgccacuacauacauugcc

SEQ ID NO: 225 - RNA, 25 nt, Artificial Sequence
  the sequence is synthesized
uuuacuccaaauauagcuguaaccc

SEQ ID NO: 226 - RNA, 25 nt, Artificial Sequence
  the sequence is synthesized
uuguuggguuuacuccaaauauagc

SEQ ID NO: 227 - RNA, 25 nt, Artificial Sequence
  the sequence is synthesized
uugcuguugcugucugcuaauaagg

SEQ ID NO: 228 - RNA, 25 nt, Artificial Sequence
  the sequence is synthesized
uagguaucaaaguauguccaacacg

SEQ ID NO: 229 - RNA, 25 nt, Artificial Sequence
  the sequence is synthesized
uucggugucugcaucuuccucuucc

SEQ ID NO: 230 - RNA, 25 nt, Artificial Sequence
  the sequence is synthesized
aaacguuccgaaaggguuuccuucg

SEQ ID NO: 231 - RNA, 25 nt, Artificial Sequence
  the sequence is synthesized
augucuguaugccauguucccuugc

SEQ ID NO: 232 - RNA, 25 nt, Artificial Sequence
  the sequence is synthesized
aagcagugaguagguucuguauucc

SEQ ID NO: 233 - RNA, 25 nt, Artificial Sequence
  the sequence is synthesized
aucagucauauaauacacacugucc

SEQ ID NO: 234 - RNA, 25 nt, Artificial Sequence
  the sequence is synthesized
uuaacaagcugaguagcggauaccg

SEQ ID NO: 235 - RNA, 25 nt, Artificial Sequence
  the sequence is synthesized
aauguauuauaggcguaguguuacc

SEQ ID NO: 236 - RNA, 25 nt, Artificial Sequence
  the sequence is synthesized
uaugguauguuacagucaguauucc

SEQ ID NO: 237 - RNA, 25 nt, Artificial Sequence
  the sequence is synthesized
aagcugaguagcggauaccgugucg

SEQ ID NO: 238 - RNA, 25 nt, Artificial Sequence
  the sequence is synthesized
uuuaacaagcugaguagcggauacc

SEQ ID NO: 239 - RNA, 25 nt, Artificial Sequence
  the sequence is synthesized
uagcuguuuaacaagcugaguagcg

SEQ ID NO: 240 - RNA, 25 nt, Artificial Sequence
  the sequence is synthesized
aaguggguugacagguccacaaugc

SEQ ID NO: 241 - RNA, 25 nt, Artificial Sequence
  the sequence is synthesized
uagguguagcugcaccgagaagugg

SEQ ID NO: 242 - RNA, 25 nt, Artificial Sequence
  the sequence is synthesized
uaguguuaccacuacagaguuuccg

SEQ ID NO: 243 - RNA, 25 nt, Artificial Sequence
  the sequence is synthesized
auacgcacacauacagacagauggc

SEQ ID NO: 244 - RNA, 25 nt, Artificial Sequence
  the sequence is synthesized
uauacacaaauaccaauacccaugc

SEQ ID NO: 245 - RNA, 25 nt, Artificial Sequence
  the sequence is synthesized
auauacugugaaugcuguggcaggg

SEQ ID NO: 246 - RNA, 25 nt, Artificial Sequence
  the sequence is synthesized
auacauauacugugaaugcuguggc

SEQ ID NO: 247 - RNA, 25 nt, Artificial Sequence
  the sequence is synthesized
aauacauauacugugaaugcugugg

SEQ ID NO: 248 - RNA, 25 nt, Artificial Sequence
  the sequence is synthesized
auguauaugcaauaguaacaugggc

SEQ ID NO: 249 - RNA, 25 nt, Artificial Sequence
  the sequence is synthesized
aagucaguuaccgaagcccguuugc

SEQ ID NO: 250 - RNA, 25 nt, Artificial Sequence
  the sequence is synthesized
aacgcccacccaauggaauguaccc

SEQ ID NO: 251 - RNA, 25 nt, Artificial Sequence
  the sequence is synthesized
uacccgcagauguuauaucaaaccc

SEQ ID NO: 252 - RNA, 25 nt, Artificial Sequence
  the sequence is synthesized
auacccauguguuccagauguaggg

SEQ ID NO: 253 - RNA, 25 nt, Artificial Sequence
  the sequence is synthesized
uuagccacugacacuuguugguagg

SEQ ID NO: 254 - RNA, 25 nt, Artificial Sequence
  the sequence is synthesized
uaccgcugcggguaaacauaguugc

SEQ ID NO: 255 - RNA, 25 nt, Artificial Sequence
  the sequence is synthesized
auaucaaacaagucauuguccuccg

SEQ ID NO: 256 - RNA, 25 nt, Artificial Sequence
  the sequence is synthesized
uauacaggcacaucccaagaggagg

SEQ ID NO: 257 - RNA, 25 nt, Artificial Sequence
  the sequence is synthesized
uaccaauauacuguguagaggcagg

SEQ ID NO: 258 - RNA, 25 nt, Artificial Sequence
  the sequence is synthesized
uuauuugggucagguaacugcaccc

SEQ ID NO: 259 - RNA, 25 nt, Artificial Sequence
  the sequence is synthesized
uacuaaaguccauggcaccauaucc

SEQ ID NO: 260 - RNA, 25 nt, Artificial Sequence
  the sequence is synthesized
uuauuaaacaacugggagucagagg

SEQ ID NO: 261 - RNA, 25 nt, Artificial Sequence
  the sequence is synthesized
gguagauaccacucccaguaccaau

SEQ ID NO: 262 - RNA, 25 nt, Artificial Sequence
  the sequence is synthesized
auuguuaaauugguacugggagugg

SEQ ID NO: 263 - RNA, 25 nt, Artificial Sequence
  the sequence is synthesized
auguauccaccaaacuaguaguugg

SEQ ID NO: 264 - RNA, 25 nt, Artificial Sequence
  the sequence is synthesized
uuggcagguuuagaagacguagugg

SEQ ID NO: 265 - RNA, 25 nt, Artificial Sequence
  the sequence is synthesized
uugcugucacauccacagcaacagg

SEQ ID NO: 266 - RNA, 25 nt, Artificial Sequence
  the sequence is synthesized
uucagaauagccauauccacugucc

SEQ ID NO: 267 - RNA, 25 nt, Artificial Sequence
  the sequence is synthesized
uuggcuauugcuuccaucuuccucg

SEQ ID NO: 268 - RNA, 25 nt, Artificial Sequence
  the sequence is synthesized
aucuaaacgcuuggcuauugcuucc

SEQ ID NO: 269 - RNA, 25 nt, artificial sequence
  the sequence is synthesized
aacugugcugccauaacauacuucc

SEQ ID NO: 270 - RNA, 25 nt, Artificial Sequence
  the sequence is synthesized
aagcaggaguuacaggacuaaaggg

SEQ ID NO: 271 - RNA, 25 nt, Artificial Sequence
  the sequence is synthesized
aaagcaggaguuacaggacuaaagg

SEQ ID NO: 272 - RNA, 25 nt, Artificial Sequence
  the sequence is synthesized
aaacagggccuguagguaaagcagg

SEQ ID NO: 273 - RNA, 25 nt, Artificial Sequence
  the sequence is synthesized
uauacugugcugucgcuaggccgcc

SEQ ID NO: 274 - RNA, 25 nt, Artificial Sequence
  the sequence is synthesized
auauacugugcugucgcuaggccgc

SEQ ID NO: 275 - RNA, 25 nt, Artificial Sequence
  the sequence is synthesized
uugcaaaucuaacauauauucaugc

SEQ ID NO: 276 - RNA, 24 nt, Artificial Sequence
  the sequence is synthesized
ugcagauccaacauauauucaugc

SEQ ID NO: 277 - RNA, 25 nt, Artificial Sequence
  the sequence is synthesized
uacaauauuguaaugggcucugucc

SEQ ID NO: 278 - RNA, 24 nt, Artificial Sequence
  the sequence is synthesized
acgauguuguagugggcucugucc

SEQ ID NO: 279 - RNA, 25 nt, Artificial Sequence
  the sequence is synthesized
aaaagguuacgguauuguaaugggc

SEQ ID NO: 280 - RNA, 25 nt, Artificial Sequence
  the sequence is synthesized
aaaaggucacgauguuguagugggc

SEQ ID NO: 281 - RNA, 25 nt, Artificial Sequence
  the sequence is synthesized
uagugugcccauuaacaggucuucc

SEQ ID NO: 282 - RNA, 24 nt, Artificial Sequence
  the sequence is synthesized
agggugcccaucagcaggucuucc

SEQ ID NO: 283 - RNA, 25 nt, Artificial Sequence
  the sequence is synthesized
aauuccuagugugcccauuaacagg

SEQ ID NO: 284 - RNA, 24 nt, Artificial Sequence
  the sequence is synthesized
augcccagggugcccaucagcagg

SEQ ID NO: 285 - RNA, 25 nt, Artificial Sequence
  the sequence is synthesized
auggggcacacaauuccuagugugc

SEQ ID NO: 286 - RNA, 25 nt, Artificial Sequence
  the sequence is synthesized
auggggcacaggaugcccagggugc

SEQ ID NO: 287 - DNA, 20 bp, Artificial Sequence
  the sequence is synthesized
ggaatccatatgctgtatgt

SEQ ID NO: 288 - DNA, 20 bp, Artificial Sequence
  the sequence is synthesized
ctacgtgttcttgatgatct

SEQ ID NO: 289 - DNA, 20 bp, Artificial Sequence
  the sequence is synthesized
caacattagaacagcaatac

SEQ ID NO: 290 - DNA, 20 bp, Artificial Sequence
  the sequence is synthesized
atgatctgcaacaagacata

SEQ ID NO: 291 - DNA, 23 bp, Artificial Sequence
  the sequence is synthesized
attgcatgaatatatgttagatt

SEQ ID NO: 292 - DNA, 23 bp, Artificial Sequence
  the sequence is synthesized
cacaattcctagtgtgcccatta

SEQ ID NO: 293 - DNA, 20 bp, Artificial Sequence
  the sequence is synthesized
acacttcactgcaagacata

SEQ ID NO: 294 - DNA, 20 bp, Artificial Sequence
  the sequence is synthesized
ccatacacagagtctgaata

SEQ ID NO: 295 - DNA, 20 bp, Artificial Sequence
  the sequence is synthesized
agacagtattggaacttaca

SEQ ID NO: 296 - DNA, 20 bp, Artificial Sequence
  the sequence is synthesized
tacacagagtctgaataatg

SEQ ID NO: 297 - DNA, 17 bp, Artificial Sequence
  the sequence is synthesized
tgcatttagagccccaa

SEQ ID NO: 298 - DNA, 17 bp, Artificial Sequence
  the sequence is synthesized
cacaaaggacagggtgt

SEQ ID NO: 299 - DNA, 23 bp, Artificial Sequence
  the sequence is synthesized
attgcatgaatatatgttagatt

SEQ ID NO: 300 - DNA, 23 bp, Artificial Sequence
  the sequence is synthesized
cacaattcctagtgtgcccatta

SEQ ID NO: 301 - DNA, 17 bp, Artificial Sequence
  the sequence is synthesized
tgcatttagagccccaa

SEQ ID NO: 302 - DNA, 17 bp, Artificial Sequence
  the sequence is synthesized
cacaaaggacagggtgt

SEQ ID NO: 303 - protein, 18 aa, Artificial Sequence
  the sequence is synthesized
KHHHKHHHKHHHHKHHHK